(19) United States
(12) Patent Application Publication
(10) Pub. No.: US 2026/0201327 A1
(43) Pub. Date: Jul. 16, 2026

(54) IN VITRO CULTIVATION METHOD FOR ANTIBODY EXPRESSING CELLS

(71) Applicant: Roche Diagnostics Operations, Inc., Indianapolis, IN (US)

(72) Inventors: Janis Brockhaus, Penzberg (DE); Sebastian Buchenberg, Penzberg (DE); Caroline Dorothea Hojer, Penzberg (DE); Thomas Meier, Penzberg (DE)

(21) Appl. No.: 19/348,422

(22) Filed: Oct. 2, 2025

Related U.S. Application Data
(63) Continuation of application No. PCT/EP2024/059116, filed on Apr. 4, 2024.

(30) Foreign Application Priority Data
Apr. 5, 2023 (EP) 23166799.9

(51) Int. Cl. C12N 5/078; C07K 16/00; C12N 5/00

(52) U.S. Cl. CPC C12N 5/0634; C07K 16/00; C12N 5/0087; C07K 2317/14; C07K 2317/56; C12N 2501/2302; C12N 2501/2315; C12N 2501/2321; C12N 2501/25; C12N 2501/52

(57) ABSTRACT

The present invention relates to an in vitro method for cultivating one or more antibody expressing cell(s). The method comprises cultivating one or more antibody expressing cell(s) obtained from peripheral blood in the presence of IL-2, IL-21 and a non-cell surface presented CD40-stimulating agent and in the absence of feeder cells. Moreover, herein provided are methods for producing antibodies comprising the step of cultivating one or more antibody expressing cell(s) according to the method of the invention, a novel CD40-stimulating agent and uses therefrom as well as a cell culture medium.

### CROSS-REFERENCE TO RELATED APPLICATIONS

This application is a continuation of International PCT Application No. PCT/EP2024/0591 16 filed on Apr. 4, 2024, which claims priority to European Patent Application No. 23166799.9 filed on Apr. 5, 2023, the contents of each application are incorporated herein by reference in their entireties.

### SEQUENCE LISTING

This application incorporates by reference the material in the ST.26 XML file titled P38401-WO-Sequence-Listing.xml, which was created on Oct. 2, 2025 and is 24,625 bytes.

The present invention relates to an in vitro method for cultivating one or more antibody expressing cell(s). The method comprises cultivating one or more antibody expressing cell(s) obtained from peripheral blood in the presence of IL-2, IL-21 and a non-cell surface presented CD40-stimulating agent and in the absence of feeder cells. Moreover, herein provided are methods for producing antibodies comprising the step of cultivating one or more antibody expressing cell(s) according to the method of the invention, a novel CD40-stimulating agent and uses therefrom as well as a cell culture medium.

The development of monoclonal antibodies is commonly based on immunization of animals with the desired target antigen. Standard species are mouse, rat and hamster to generate hybridomas by fusion of splenic cells with myeloma cells (Milstein and Kohler), and rabbits. In the case of rabbits, both hybridoma (Spieker-Polet H, Sethupathi P, Yam P C, Knight K L. Proc Natl Acad Sci USA. 1995; 92(20):9348-9352) and B cell cloning (Seeber et al, PLoS One. 2014 Feb. 4; 9(2):e86184. doi: 10.1371/journal.pone.0086184) can be employed. B cell cloning requires ex vivo culturing and proliferation of primary antibody expressing cells isolated from peripheral blood. Methods for culturing B cells in the context of B cell cloning have been, for example, described in WO2011/147903(A1), WO2013/076139(A1), WO2017/167714(A1), WO2018/122147(A1), WO2018/210896(A1) and WO2019/105864(A1).

Efficient culturing and proliferation of primary antibody expressing cells obtained from peripheral blood is typically conducted in a feeder cell based system using e.g. EL4-B5 cells and in the presence of a conditioned cell supernatant (Zubler et al, 1985, J Immunol 1 Jun. 1985; 134 (6): 3662-3668). The conditioned cell supernatant is usually derived from a thymocyte cell line (thymocyte supernatant (TSN)) (Steenbakkers et al, Mol Biol Rep. 1994 March; 19(2):125-34). This type of culture using feeder cells and TSN has been commonly employed for efficient proliferation of primary antibody expressing cells obtained from peripheral blood in a single-cell format.

However, cultivation systems using feeder cells and TSN have certain shortcomings. Firstly, feeder cells need to be irradiated in order to avoid overgrowing of the antibody expressing cells. This irradiation is a cumbersome and time consuming step, which also requires specialized and expensive equipment. Secondly, the presence of feeder cells can hamper the usage of modem single cell technologies like droplet based systems, scale down processes and high throughput systems due to the fact that always a mixture of feeder and antibody expressing cells is required. Irradiated feeder cells can also complicate the usage of automation systems because these cells have a short lifespan and the automation process has to be more complex for handling feeder cells. Finally, using feeder cells and cell-derived conditioned media such as TSN is prone to lot-to-lot variations and, thus, requires a complex and time-consuming preparation process with a high need for quality control.

In view of the above, there is a high need to address the above-mentioned shortcomings of the ex vivo culturing of primary antibody expressing cells and to thereby improve B cell cloning technologies and antibody production processes.

These shortcomings are addressed by the present invention, which is defined by the claims and the disclosure herein below.

According to a first aspect, the present invention relates to an in vitro method for cultivating one or more antibody expressing cell(s) obtained from peripheral blood. The method comprises cultivating the one or more antibody expressing cell(s) in the presence of IL-2, IL-21 and a non-cell surface presented CD40-stimulating agent (e.g. a soluble CD40-stimulating agent).

Using the cultivation method of the invention the one or more antibody expressing cell(s) obtained from peripheral blood can be proliferated, i.e. the one or more antibody expressing cell(s) may divide and increase in number during the cultivation.

Accordingly, the method of the first aspect may be an in vitro method for proliferating one or more antibody expressing cell(s) obtained from peripheral blood, said method comprising proliferating the one or more antibody expressing cell(s) by cultivating the one or more antibody expressing cell(s) in the presence of IL-2, IL-21 and a non-cell surface presented CD40-stimulating agent.

As demonstrated by the appended Examples the present inventors have surprisingly found that antibody expressing cells, in particular antibody secreting cells (e.g. plasmablasts) obtained from peripheral blood (e.g. rabbit peripheral blood) can be cultured and proliferated without losing the capability to produce antibodies using the method according to the first aspect, even in the absence of feeder cells (e.g. EL4B5) and conditioned cell supernatant (e.g. TSN). In particular, it has been demonstrated that by adding IL-2, IL-21 and a non-cell surface presented CD40-stimulating agent (e.g. soluble CD40-L) to the culture medium, antibody expressing cells obtained from peripheral blood could be proliferated at least as efficient as in the presence of feeder cells and TSN. Very surprisingly, the appended examples also demonstrated that even a single antibody expressing cell obtained from peripheral blood could be successfully cultured and proliferated efficiently using the method of the invention in the absence of feeder cells and TSN, and consequently also in the absence of any cell-cell contacts. Cell-cell contacts were believed to be important for efficient proliferation (see Zuber et al. 1985, supra).

The surprising finding that the commonly used feeder cells and TSN can be replaced without losing substantial cultivation and proliferation capacity, solves the shortcomings associated with feeder cells and cell derived conditioned medium (e.g. TSN), as described above. Thus, the cultivation process becomes easier to handle, more robust and less time and resource consuming. Another important advantage is that in the absence of feeder cells smaller cultivation vessels can be used, which in turn allows usage of higher multiplexed systems, e.g. 1536 well plates or 384 well plates instead of 96 well plate. In other words, automation and high throughput approaches become easier to apply. Similarly, applicability of automated systems becomes possible, as these approaches can be severely hampered by the requirement for feeder cells.

In the method of the first aspect, the cultivation step is conducted in the absence of feeder cells, in particular in the absence of feeder cells for cultivation of antibody expressing cells (e.g. EL4B5 cells as frequently used for this purpose). In other words, the cultivating step does preferably not comprise co-cultivation with feeder cells, in particular feeder cells for cultivation of antibody expressing cells, such as EL4B5. Other exemplary but non-limiting examples for feeder cells for cultivation of antibody-secreting cells are: 3T3 cells (J. Huanget al., 2013, Cells. Nat. Protoc. 8, 1907-1915) and CD40-L expressing mammalian cells such as described in WO2013076139.

Further, in the method of the first aspect, the cultivation step is conducted in the absence of any conditioned cell supernatant, such as in particular thymocte supernatant (TSN). “Conditioned cell supernatant” relates to a medium supernatant that is obtained by culturing cells secreting cytokines stimulating survival and proliferation of antibody expressing cells obtained from peripheral blood (e.g. plasmablasts) in a medium for a defined time. The medium supernatant is removed from the cells secreting cytokines stimulating survival and proliferation of antibody expressing cells obtained from peripheral blood (e.g. plasmablasts) after the defined time and comprises cytokines and substances that facilitate survival and proliferation of antibody expressing cells ex vivo. A preferred example for a conditioned cell supernatant is TSN. TSN can be generated as described in Steenbakkers et al, Mol Biol Rep. 1994 March; 19(2):125-34, which is incorporated herein in its entirety. In brief, TSN may be obtained as follows: Thymocytes obtained from 4-6 weeks old rabbits are cultured in a cell density of about 2×10⁷ cells/ml in the presence of 5pg/ml PHA-M (Phytohemagglutinin M) and 50 ng/ml PMA (Phorbol myristate acetate) for 24 hours. This cultivation may be performed in the following medium: RMPI 1640, supplemented with 10% FCS, 0.29 g/l glutamine, 2 mM Na-Pyruvat, 5 mM Hepes and 2.5 μM β-mercaptoethanol. Subsequently, the supernatant (=TSN) is obtained by centrifugation and sterile filtration.

In embodiments of the method of the first aspect, the cultivation of the antibody expressing cells obtained from peripheral blood is performed in the presence of IL-2, IL-21 and IL-15. As shown in the appended examples, the additional presence of IL-15 further facilitates proliferation of the antibody expressing cell(s).

In embodiments of the method of the first aspect, the cultivation of the antibody expressing cells obtained from peripheral blood is performed in the presence of IL-2, IL-21 and B-cell activating factor (BAFF). BAFF is in particular soluble BAFF in the context of the invention. As shown in the appended examples, the additional presence of BAFF further facilitates proliferation of the antibody expressing cell(s).

In particularly preferred embodiments, the cultivation of the antibody expressing cells obtained from peripheral blood is performed in the presence of IL-2, IL-15, IL-21 and BAFF. As shown in the appended examples, this combination of cytokines facilitates proliferation of the antibody expressing cell(s) the best of all conditions tested herein.

In embodiments, any one or all of IL-2, IL-15, IL-21 and BAFF may be recombinantly produced. Recombinantly produced IL-2, IL-15, IL-21 and/or soluble BAFF can be commercially purchased as also discussed in the appended Examples below.

The IL-2, IL-21, IL-15 and/or BAFF as used herein may be from the same or a different species origin as the antibody expressing cells, provided that they have the corresponding cultivating and proliferating activity on the antibody expressing cells as described herein. As demonstrated in the appended Examples, human IL-2, human IL-21, human IL-15 and human BAFF could be successfully used in conjunction with rabbit antibody expressing cells. In one embodiment, the antibody expressing cells obtained from peripheral blood are rabbit antibody expressing cells and either one of IL-2, IL-21, IL-15 and BAFF is independently selected from human or rabbit.

In one embodiment, the antibody expressing cells are rabbit antibody expressing cells obtained from peripheral blood and each of IL-2, IL-21, IL-15 and BAFF are human. In one embodiment, the antibody expressing cells are rabbit antibody expressing cells obtained from peripheral blood and each of IL-2, IL-21, IL-15 and BAFF are rabbit.

In the appended examples, it has been found that certain compounds (e.g. cytokines) may be detrimental for cultivating and in particular proliferating antibody expressing cells. Thus, in embodiments, the cultivating of the one or more antibody expressing cell(s) is preferably performed in the absence of one or more selected from: Inducible co-stimulator (ICOS), 4-1BB (also known as CD137), Phorbol myristate acetate (PMA), and IL-4.

In embodiments, the one or more antibody expressing cell(s) is/are primary. “Primary” as used herein means that the cells are obtained from previously obtained peripheral blood or previously obtained peripheral blood mononuclear cells (PBMCs) directly, i.e. without further modification (such as immortalization by any means), for ex vivo cultivation. In preferred embodiments, primary means that the antibody expressing cells are not frozen after being obtained from peripheral blood. Thus, the one or more antibody expressing cells may be non-immortalized. In embodiments, primary antibody expressing cell(s) may be obtained 24 hours or less, in particular 12 hours or less, in particular 8 hours or less, in particular 4 hours or less, in particular 1 hour or less before the cultivating is started.

As demonstrated in the appended Examples, the cultivation step of the method of the first aspect can efficiently proliferate antibody expressing cells in small cell numbers and even in a single cell culture system (i.e. in the latter even initially in absence of any cell-cell contacts). Thereby culturing systems with small volumes (e.g. 1536 well plates or 384 well plates) with a high suitability for high throughput screenings can be used.

The one or more antibody expressing cell(s) may be 100 or less antibody expressing cells, preferably 75 or less antibody-secreting cell(s), even more preferably 50 or less antibody expressing cell(s) and even more preferably 20 or less antibody expressing cell(s) during cultivation. In a particularly preferred embodiment, the one or more antibody expressing cell(s) may be a single antibody expressing cell. This single antibody expressing cell may be cultured in a single cell format and may be proliferated during the cultivating. Single cell culturing and proliferation of antibody expressing cells is very important for B cell sorting methods employed for screening of antibodies directed against an antigen of interest. Having single cells allows for easy and unambiguous determination of the coding sequence for the antibody produced by the single antibody expressing cell.

In embodiments, the cultivation of the method of the first aspect may be in a medium volume of at most 190 μl, at most 100 μl, and preferably at most 70 μl. In certain embodiments the volume may be at most 10 μl. As the present method does not require feeder cells, it is a particular advantage of the method of the invention that such small volumes can be used for the cultivation. In particular, the method of the invention may be used with 384-well, 1536-well plates and droplet technologies, which is difficult or even impossible with using feeder cells.

The one or more antibody expressing cells used herein are obtained from peripheral blood. “Obtained from peripheral blood” includes cells directly obtained from fresh previously isolated peripheral blood (i.e. primary cells) but also cells derived from previously obtained and stored (e.g. frozen) Peripheral Blood Mononuclear Cells (PBMCs). In certain embodiments, the antibody expressing cells employed in the method according to the first aspect of the invention are directly obtained from peripheral blood, i.e. no frozen storage of blood or PBMCs is involved.

In embodiments, the method according to the first aspect may comprise isolating the one or more antibody expressing cells from peripheral blood and/or previously isolated PBMCs ex vivo. “Isolating the one or more antibody expressing cells” means that the cells are enriched such that they are substantially free of non-antibody expressing cells. In embodiments, “substantially free of non-antibody expressing cells” means that 20% or less, preferably 10% or less, and most preferably 5% or less of the cells obtained by the isolation are non-antibody expressing cells. In embodiments, “isolating” or “isolated” may be referred to as “enriching” or “enriched”, respectively.

Ex vivo isolation of the one or more antibody expressing cells from peripheral blood (i.e. obtaining antibody expressing cells from peripheral blood) may comprise (i) isolating PBMCs from peripheral blood and (ii) isolating the one or more antibody expressing cell(s) from the isolated PBMCs. In embodiments, PBMCs may be obtained and the one or more antibody expressing cell(s) may be isolated from the PBMCs.

“Peripheral blood mononuclear cells (PBMCs)” are any peripheral blood cells having a round nucleus. These cells consist of lymphocytes (T cells, B cells, NK cells) and monocytes, whereas erythrocytes and platelets have no nuclei, and granulocytes (neutrophils, basophils, and eosinophils) have multi-lobed nuclei. Accordingly, PBMCs essentially consist of lymphocytes and monocytes; i.e. are lymphocytes and monocytes enriched from previously obtained peripheral blood. As will be appreciate by a skilled person, PBMCs are substantially free of other cells but may comprise a minor amount of other cells as the methods used for isolating PBMCs will hardly lead to a 100% purity. In embodiments, PBMCs consist to at least 80%, preferably at least 85%, even more preferably 90%, even more preferably 95% and most preferably at least 99% of mononuclear peripheral blood cells (i.e. lymphocytes and monocytes).

“Peripheral blood” is the blood circulating throughout the body. The cellular components that could be isolated from human peripheral blood include erythrocytes (red blood cells), leukocytes (white blood cells), and thrombocytes (platelets).

In the context of the present invention the antibody expressing cells are obtained ex vivo from peripheral blood. In particular embodiments, the peripheral blood is obtained from a previously immunized animal (e.g. rabbit, rat, mouse, sheep or hamster), wherein the animal has been immunized with an antigen of interest. In embodiments, the peripheral blood may be obtained 4 days after immunization until 15 days after immunization or the most recent boost. In one embodiment, the peripheral blood may be obtained after 4 days until at most 9 days after immunization or the most recent boost.

Methods for isolating PBMCs from a peripheral blood sample are well known in the art. These cells can, for example, be extracted from blood using ficoll, a hydrophilic polysaccharide that separates layers of blood, and gradient centrifugation, which will separate the blood into a top layer of plasma, followed by a layer of PBMCs and a bottom fraction of polymorphonuclear cells (such as neutrophils and eosinophils) and erythrocytes. Non-limiting examples for such methods are described in Kleiveland C. R. (2015) Scan. J. Clin. Lab. Invest. 1968, 21 (Suppl. 97): 77-89, which is herein incorporated in its entirety. In an exemplary method, PBMCs may be obtained from peripheral EDTA blood as described in the following. EDTA blood may be diluted 1:1 with PBS (e.g. Gibco, 10010-015) and applied to one or more Leucosep™ Tubes (e.g., Greiner bio-one, 227288), e.g. at a volume of 20-30 ml. The tubes may subsequently be centrifuged, e.g. at 800 g for 15 min without terminal break. The interphase formed in the tube(s) may then be collected in a collection tube (e.g. Falcon, 352075) with PBS (e.g. 130 ml) and the cells may be pelleted by centrifugation (e.g. for 15 min). The cells may then be resuspended in solution or medium. To determine cell numbers, e.g. C-Chip™ Fuchs-Rosenthal Counting Chambers (NanoEnTek, DHC-FO1) and Trypan Blue may be used.

Obtaining/Isolating one or more antibody expressing cells from peripheral blood and/or PBMCs may comprise isolating/enriching cells expressing a membrane-bound antibody (B cell receptor) (or a plurality of copies thereof). For instance, enrichment methods such as MACS based techniques or FACS based cell sorting may be used for enriching the antibody expressing cells, e.g. by selecting/enriching for cells express surface antibodies such as of subtype IgG, IgA or IgM (e.g. IgG).

For instance, IgG-positive antibody expressing cells may be isolated by staining the cells with a dye-labeled anti-IgG antibody and sorting IgG positive cells. For obtaining antibody expressing cells, cells may additionally also be stained with 7-Aminoactinomycin D (7-AAD), a dye that can discriminate alive cells from apoptotic cells. In preferred embodiments, cells with an intermediate cell size (FSC^{int}) as analyzed by the flow cytometer may be selected to obtain antibody expressing cells. In embodiments, cells with an intermediate positive for IgG (IgG^{int}) as analyzed by the flow cytometer may be selected as antibody expressing cells. In embodiments the antibody expressing cells obtained from peripheral blood are plasmablasts. Plasmablasts have an intermediate staining for IgG (IgG^{int}) and an intermediate cell size (FSC^{int}) in PBMCs as analyzed by the flow cytometer and can be selected accordingly. Plasmaplasts distinguish from memory B cells in that they are lower in IgG expression and bigger in size (FSC). In embodiments the antibody expressing cells are comprise plasmablasts but not memory B cells. In embodiments, the antibody expressing cells are enriched for plasmablasts vs. memory B cells.

In flow cytometry, the light scattered by cells can be measured by two optical detectors: forward scatter (FSC) that detects scatter along the path of the laser, and side scatter (SSC) which measures scatter at a ninety-degree angle relative to the laser.

FSC intensity is proportional to the diameter of the cell, and is primarily due to light diffraction around the cell. FSC signal can be used for the discrimination of cells by size. SSC, on the other hand, is from the light refracted or reflected at the interface between the laser and intracellular structures, such as granules and nucleus. SSC provides information about the internal complexity (i.e. granularity) of a cell.

In embodiments, the antibody expressing cells cultivated in the context of the invention are plasmablasts. In embodiments the antibody expressing cells cultivated in the context of the invention are not memory B-cells.

Antibody expressing cells (e.g. plasmablasts) are to be isolated, cells may further be selected/enriched for cells binding a respective antigen of interest on the cell surface (i.e. antigen specific antibody expressing cells). Optionally, antigen-specific antibody expressing cells may be selected for not binding one or more potential cross-reacting structures. Antigen-specific antibody expressing cells are preferably obtained from peripheral blood obtained from blood of a previously immunized animal (e.g. rabbit, mouse, rat, sheep, hamster), in particular rabbit, which was immunized with the antigen of interest.

Antigen-specific antibody expressing cells (such as plasmablasts) may be enriched from peripheral blood/PBMCs via binding to the antigen of interest, e.g. with MACS or FACS based techniques (e.g. via staining with a dye labeled antigen of interest). Further, cells may be stained with an antibody directed against the antibody class of interest (e.g. IgG, IgA or IgM) to be able to select antibody expressing via FACS sorting. For instance, IgG-positive antibody expressing cells may be isolated by staining the cells with a dye-labeled anti-IgG antibody and sorting IgG positive cells. Cells may additionally also be stained with 7-Aminoactinomycin D (7-AAD), a dye that can discriminate alive cells from apoptotic cells. In preferred embodiments, cells with an intermediate cell size (FSC^{int}) as analyzed by the flow cytometer may be selected. In embodiments, cells with an intermediate positive for IgG (IgG^{int}) as analyzed by the flow cytometer may be selected. Plasmablasts are have typically an intermediate staining for IgG (IgG^{int}) and an intermediate cell size (FSC^{int}) as analyzed by the flow cytometer and can be selected accordingly. Plasmaplasts distinguish from memory B cells in that they are lower in IgG expression and bigger in size (FSC). In embodiments the antibody expressing cells cultivated in the context of the invention are plasmablasts. In embodiments the antibody expressing cells cultivated in the context of the invention are not memory B-cells.

Accordingly, in embodiments, antibody expressing cells are cells obtainable or obtained from peripheral blood (e.g. PBMCs obtained from peripheral blood) by enriching cells with an intermediate size (FSCi^{int}) expressing an antibody (e.g. an antibody such as IgG, IgA or IgM) specific for a certain antigen on their surface. In a particular embodiment antibody expressing cells are IgG expressing cells, even more preferably intermediate IgG expressing cells (most preferably plasmablasts). In embodiments, the antibody expressing cells may be IgG-positive. In embodiments, the antibody expressing cells may be IgM-positive. In embodiments, the antibody expressing cells may be IgA-positive. “IgX” (i.e. IgG, IgM or IgA) positive means that the cells express the “IgX” on the cell surface and optionally secret the same. Accordingly, in embodiments the antibody expressing cells express and/or secret IgG, IgM or IgA.

Additionally or alternatively, isolating or enriching one or more antibody expressing cells from peripheral blood and/or PBMCs may comprise isolating/enriching cells expressing one or more surface markers. Such surface markers may in particular be selected from the group consisting of CD19 and CD43 or a combination thereof. In other words, obtaining antibody expressing cells (e.g. antibody secreting cells such as plasmablasts) may comprise selection of cells by labeling surface markers, said cells being CD19-positive and/or CD43-positive. Plasmablasts are known to express CD19 and CD43 on their cell surface.

In particular embodiments of the method according to the first aspect, the one or more antibody expressing cells may comprise or consist of antigen-binding antibody expressing cells. An “antigen-binding antibody expressing cell” as used herein, is a cell that expresses an antibody binding to a particular antigen of interest. Accordingly, “antigen-specific antibody expressing cells” are cells that each express an antibody binding the same antigen of interest. Thus, in embodiments, the one or more antibody expressing cells may, each independently, express an antibody directed against the same antigen of interest.

In particular embodiments of the method according to the first aspect, the one or more antibody expressing cells may comprise or consist of antigen-specific antibody expressing cells. An “antigen-specific antibody expressing cell” as used herein, is a cell that expresses an antibody specifically binding to a particular antigen of interest. Accordingly, “antigen-specific antibody expressing cells” are cells that each express an antibody specifically binding the same antigen of interest. Thus, in embodiments, the one or more antibody expressing cells may, each independently, express an antibody directed against the same antigen of interest.

Isolating one or more antigen-specific antibody expressing cells from peripheral blood and/or PBMCs may comprise selecting/enriching cells expressing an antibody (or a plurality of copies thereof) binding (in embodiments specifically binding) to the antigen of interest. The enrichment/selection may be before or after the isolation or enrichment of the antibody expressing cells from peripheral blood or PBMCs. Selecting antigen-specific antibody expressing cells may additionally comprise steps of removing cells that interact with potential cross reactive compounds.

An exemplary method for selecting antigen-specific cells may comprise providing beads having the antigen of interest attached thereto, binding the cells expressing an antibody binding to the antigen of interest to said beads, separating the beads from the free cells and releasing the antigen-specific antibody expressing cells from the beads. Optionally beads may be magnetic beads. For instance, MACS technologies may be employed. In alternative methods, labelled antigen of interest may be used to stain cells and stained cells may be selected, e.g. by FACS sorting. For instance the label may be a fluorescence dye and the sorting may be achieved by FACS. Exemplary methods are also described in the appended examples.

In embodiments, the one or more antibody expressing cell(s) are obtainable or obtained by a method comprising (i) isolating PBMCs from an obtained peripheral blood sample; (ii) isolating from the isolated PBMCs one or more antibody expressing cell(s), preferably one or more antibody expressing cell(s) binding to an antigen of interest. The individual steps may be as in any of the above defined embodiments thereof. Optionally, steps enriching cells expressing one or more surface markers of antibody expressing cells may be conducted prior to or after step ii) (see above).

In embodiments, the method of the first aspect may comprise (i) isolating PBMCs from an obtained peripheral blood sample; (ii) isolating from the isolated PBMCs one or more antibody expressing cell(s), preferably one or more antibody expressing cell(s) binding to an antigen of interest. The individual steps may be as in any of the above defined embodiments thereof.

In embodiments, the antibody expressing cells used in the context of the present invention are cells that are obtained or obtainable from peripheral blood via an isolation method comprising:
i) Isolating PBMCs from peripheral blood
ii) Isolating from said PBMCs cells that are: a) IgG positive (e.g. IgG^{int}) and b) have an intermediate cell size (FSC^{int}) preferably using flow cytometry such as FACS sorting.

In embodiments, in ii) the cells may have an intermediate IgG expression. In embodiments, the cells isolated in ii) are selected to be negative in a staining for apoptosis (e.g., 7AAD staining), i.e. said cells are non-apoptotic. In embodiments, CD19-positive and/or CD43-positive cells may be selected.

In embodiments, the antibody expressing cells used in the context of the present invention are expressing antibodies specifically binding to an antigen of interest (i.e. antigen-specific antibody expressing cells, which may be obtained or obtainable from peripheral blood via an isolation method comprising:
i) Isolating PBMCs from peripheral blood
ii) Isolating from the PBMCs, cells binding to an antigen of interest at their cell surface (e.g. via MACS or flow cytometry using a labeled antigen of interest)
iii) Isolating cells that are: a) IgG positive (e.g. IgG^{int}) and b) have an intermediate cell size (FSC^{int}) preferably using flow cytometry such as FACS sorting.

In embodiments, in iii) the cells may have an intermediate IgG expression. In embodiments, the cells isolated in iii) are selected to be negative in a staining for apoptosis (e.g., 7AAD staining), i.e. said cells are non-apoptotic. In embodiments, CD19-positive and/or CD43-positive cells may be selected.

In embodiments, antibody expressing cells obtained from peripheral blood as used herein are B cells obtained from peripheral blood having an intermediate cell size and an intermediate IgG expression. Preferably, said B cells are antigen specific, i.e. have been enriched for cells binding a certain antigen of interest.

In specific embodiment, the antibody expressing cells obtained from peripheral blood are plasmablasts obtained from peripheral blood (e.g. primary plasmablasts, such as rabbit plasmablasts). Plasmablasts are obtained or obtainable by isolating PBMCs and enriching therefrom viable cells with an intermediate IgG expression and intermediate cell size (FSC^{int}) using FACS sorting.

In specific embodiments, the plasmablasts obtained from peripheral blood are antigen specific plasmablasts, i.e. plasmablasts that express antibodies directed to the same antigen of interest. Such plasmablasts are obtainable or obtained by selecting plasmablasts binding to an antigen of interest. Notably, the enrichment for antigen binding can be conducted on the PBMC level and be followed by plasmablast isolation or after isolation of plasmablasts from PBMCs. Plasmablasts are obtained or obtainable from peripheral blood by i) isolating PBMCs and ii) isolating from said PBMCs plasmablasts by isolating cells that are a) IgG positive (with intermediate IgG staining if using FACS) and have an intermediate cell size (FSCint) preferably using FACS sorting. Corresponding methods are disclosed in the appended examples and an exemplary flow cytometry blot showing a representative gating strategy for obtaining plasmablasts is shown in FIG. 9.

The one or more antibody expressing cell may be cultivated in a culture vessel. For instance, when using single cell culturing, a single antibody expressing cell may be cultured in a well of a 96-well culture plate or a 384-well plate.

The antibody expressing cell(s) obtained from peripheral blood, as used herein, may be or comprise antibody secreting cell(s) present in peripheral blood. These antibody secreting cells are characterized in that they secret antibody into the culture medium.

Antibody secreting cell(s) obtainable or obtained from peripheral blood are preferably plasmablasts in the context of the present invention. Accordingly, in embodiments the antibody expressing cell(s) obtained from peripheral blood, as used herein, may be or comprise plasmablasts.

Antibody secreting cells can be obtained as follows: (i) isolating antibody expressing cells by any of the methods described above (including any combination of enrichment steps) and (ii) cultivating the antibody expressing cells (e.g. with a method according to the first aspect of the invention) and (iii) identify antibody secreting cells by evaluating the presence of the respective class of antibody (e.g. IgG, IgM or IgA) in the culture medium.

As mentioned above, the method of the first aspect of the invention involves cultivating antibody expressing cells in the presence of a non-cell surface presented CD40-stimulating agent. “Non-cell surface presented CD40-stimulating agent” means that the CD40-stimulating agent is not presented by a cell, in particular on a cell surface. Previously published cultivation methods for antibody expressing cells, in particular B cells from peripheral blood have typically used feeder cells expressing CD40-L on the cell surface in conjunction with cytokine mixtures.

The present inventors have surprisingly found that cultivation and proliferation of antibody expressing cells obtained from peripheral blood can be achieved in the absence of feeder cells by using a non-cell presented CD-40-stimulating agent, such as in particular a soluble CD-40 ligand. This means in a single cell culturing system that the antibody expressing cells (e.g. plasmablasts) obtained from peripheral blood are cultivated and proliferated without any cell-cell contacts and without any feeder cells. This provides a lot more flexibility for culturing, including the possibility to reduce the culture vessel volume and/or to use fully automatized screening technologies.

A “non-cell presented CD40-stimulating agent” may be soluble CD40-stimulating agent or a CD40-stimulating agent coated to a surface (e.g. inner surface of a culture vessel), wherein said surface is not a cell surface. “Soluble CD40-stimulating agent” means that the CD40-stimulating agent is not presented on a cell surface or an artificial surface and is soluble in the culture medium used.

A “CD40-stimulating agent coated to a surface” is a CD40-stimulating agent, such as a CD40 ligand that is attached (covalently or non-covalently) to a surface other than a cell surface. For example, a CD40-stimulated agent coated to a surface may be a CD40-stimulating agent that is coated on the inner surface of the culture vessel used for cultivation of the antibody expressing cells.

A non-limiting example for a soluble CD40-stimulating agent is an antibody that mimics the binding of CD40-ligand (CD40-L) to CD40 and induces a singling cascade as triggered by CD40-L and CD40 interaction in the CD40 expressing cell. A non-limiting example for such an antibody are mAb G28.5 (Ledbetter J A et al., Circ Shock. 1994 October; 44(2):67-72. PMID: 7743602) and mAb 89 (Bjorck P et al., Immunology. 1994 November; 83(3):430-7. PMID: 7530692. Other non-limiting examples for soluble CD40-stimulating agents are soluble CD40 ligand constructs (e.g. MegaCD40-L from Enzo, see E. Marasco, et al.; Eur. J. Immunol. 47, 131 (2017), Application(s): Human PBMC culture) as known in the art or as newly provided herein. In preferred embodiments, a soluble CD40-L (construct) as provided herein and/or employed in the appended Examples are used. Preferrably soluble CD40 ligands are recombinantly produced.

In particularly preferred embodiments of the invention, the soluble CD40-stimulating agent is a soluble CD40 ligand (i.e. CD40-L). A “soluble CD40 ligand” is soluble in medium and is a non-membrane anchored CD40-L protein or fragment thereof that is capable of binding to and stimulating CD40 in the antibody expressing cells, i.e. mimics the natural occurring CD40-L and CD40 interaction. Typically, a soluble CD40 ligand is recombinantly expressed.

The non-cell presented CD40-stimulating agent (e.g. soluble CD40-L) as used herein may comprise a CD40L domain.

In embodiments, the non-cell presented CD40-stimulating agent (e.g. soluble CD40-L) comprises a CD40-L domain (e.g. a 18 kDa CD40-L domain) of SEQ ID NO: 1 or an amino acid sequence having at least 75%, 80%, 90%, 95% or 99% sequence identity thereto. This CD40-L domain has been shown to be sufficient for CD40 stimulation and is conserved to a sequence identity of more than 75% between rabbit and human, mouse or rat. The sequence of SEQ ID NO: 1 is a rabbit sequence. Thus, in embodiments, the non-cell presented CD40-stimulating agent (e.g. soluble CD40-L) may comprise SEQ ID NO: 1 or a sequence corresponding thereto from other species (e.g. human, mouse, rat, hamster or sheep). In embodiments, the CD40-stimulating agent (e.g. soluble CD40-L) may comprise any one of SEQ ID Nos: 1, 2, 3, 4, 5 or 6 or a sequence having at least 75% sequence identity thereto. In embodiments, the CD40-stimulating agent (e.g. soluble CD40-L) may comprise any one of SEQ ID Nos: 1, 2, 3, 4, 5 or 6 or a sequence having at least 80% sequence identity thereto. In embodiments, the CD40-stimulating agent (e.g. soluble CD40-L) may comprise any one of SEQ ID Nos: 1, 2, 3, 4, 5 or 6 or a sequence having at least 85% sequence identity thereto. In embodiments, the CD40-stimulating agent (e.g. soluble CD40-L) may comprise any one of SEQ ID Nos: 1, 2, 3, 4, 5 or 6 or a sequence having at least 90% sequence identity thereto. In embodiments, the CD40-stimulating agent (e.g. soluble CD40-L) may comprise any one of SEQ ID Nos: 1, 2, 3, 4, 5 or 6 or a sequence having at least 95% sequence identity thereto. In embodiments, the CD40-stimulating agent (e.g. soluble CD40-L) may comprise any one of SEQ ID Nos: 1, 2, 3, 4, 5 or 6 or a sequence having at least 99% sequence identity thereto.

In embodiments, the CD40-stimulating agent (e.g. soluble CD40-L) comprises the CD40-L domain, e.g., of SEQ ID NO: 1, 2, 3, 4, 5 or 6 or a sequence with a sequence identity a defined above, but no other CD40-L specific domains or sequences. As demonstrated in the appended Examples, using the 18 kDa CD40-L domain facilitated proliferation of antibody expressing cells, in particular plasmablasts, better than using the entire ectodomain (extracellular domain) of CD40-L (see SEQ ID NO: 7 for corresponding rabbit sequence). Thus, in embodiments, the CD40-stimulating agent (e.g. soluble CD40-L) as used herein does not comprises any other CD40-L ectodomain sequences other than the CD40-L 18 kDa domain (e.g. of SEQ ID NO: 1, 2, 3, 4, 5 or 6 or a sequence with a sequence identity a defined above). CD40-L ectodomain has an amino acid sequence as depicted in SEQ ID NO: 7.

In embodiments, the CD40-stimulating agent (e.g. soluble CD40-L) as used herein may comprise the amino acid sequence of SEQ ID NO: 1, 2, 3, 4, 5 or 6 or a variant thereof with CD40-L activity having at least 75%, at least 80%, at least 85%, at least 90%, at least 95% or at least 99% sequence identity thereto.

The CD40-stimulating function of a CD40-stimulating agent, in particular a soluble CD40-L is facilitated by multimerization of the CD40-L domains. As demonstrated in the appended Examples hexameric soluble CD40-L constructs surprisingly facilitate cultivation and proliferation of antibody expressing cells with better efficacy as trimeric soluble CD40-L constructs. Accordingly, in embodiments, the CD40-L construct is at least trimeric or more than trimeric. In an even more preferred embodiment, the CD40-L construct is at least tetrameric, more preferably at least hexameric or hexameric.

To achieve a multimerization of a CD40-stimulating agent (e.g. soluble CD40-L), the CD40-stimulating agent (e.g. soluble CD40-L) may comprise a multimerization domain, in particular an artificially introduced multimerization domain (i.e. a multimerization domain introduced by genetic engineering). Multimerization domains are well known in the art.

Multimerization domains leading to different stoichiometry of multimerization are known in the art. In principle, any multimerization domain resulting at least in a dimeric, preferably an at least trimeric and even more preferably at least a hexameric CD40-stimulating agent (e.g. soluble CD40-L) can be used. Additional degree of multimerization may occur by multimerization caused by the CD40-L domain, e.g. the 18 kDa domain itself. The degree of multimerization of CD40-stimulating agent (e.g. soluble CD40-L) can be analyzed by HPLC (e.g. using SEC-MALS) analyses. Corresponding examples of such methods are employed and described in the appended Examples. The multimerization domain in the context of the invention is selected such that the CD40-L activity of a fusion protein is maintained, i.e. the activity to stimulate the CD40 receptor is not negatively affect. The person skilled in the art is well aware of multimerization domains that can be selected. Exemplary but non-limiting multimerization domains are an Fc domain (e.g. of an IgG antibody), a leucine zipper domain (LZ) and a C4b domain. In embodiments an Fc domain, in particular an IgG Fc domain, in particular a human IgG Fc domain and in particular a human IgG1 Fc domain, may be used as multimerization domain.

An exemplary C4b sequence that may be used in the context of the invention is SEQ ID NO: 8 or a variant thereof having at least 80%, at least 85%, at least 90%, at least 95% or at least 98% sequence identity to SEQ ID NO: 8, said variant showing the same multimerization as SEQ ID NO: 8 if used in the CD40-stimulating agent (e.g. soluble CD40-L).

An exemplary leucine zipper sequence that may be used in the context of the invention is SEQ ID NO: 9 or a variant thereof having at least 80%, at least 85%, at least 90%, at least 95% or at least 98% sequence identity to SEQ ID NO: 9, said variant showing the same multimerization degree (e.g. trimeric) as SEQ ID NO: 9 if used in the CD40-stimulating agent (e.g. soluble CD40-L). As demonstrated in the appended Examples, this leucine zipper achieved a trimerization of the CD40-L construct.

Another exemplary leucine zipper sequence that may be used in the context of the invention is SEQ ID NO: 10 or a variant thereof having at least 80%, at least 85%, at least 90%, at least 95% or at least 98% sequence identity to SEQ ID NO: 10, said variant showing the same multimerization degree (e.g. trimeric) as SEQ ID NO: 10 if used in the CD40-stimulating agent (e.g. soluble CD40-L). As demonstrated in the appended Examples, this leucine zipper achieved a trimerization of the CD40-L construct.

Exemplary leucine zipper are also described in Burkhardt et al., Cancer Immunol Immunother. 2013 February; 62(2):347-57. doi: 10.1007/s00262-012-1331-4. Epub 2012 Aug. 25.

An exemplary IgG Fc sequence that may be used in the context of the invention is SEQ ID NO: 11 or a variant thereof having at least 80%, at least 85%, at least 90%, at least 95% or at least 98% sequence identity to SEQ ID NO: 11, said variant showing the same multimerization (e.g. hexameric) as SEQ ID NO: 11 if used in the CD40-stimulating agent (e.g. soluble CD40-L).

As demonstrated in the appended Examples using an Fc domain (e.g. of a human IgG1) results in a soluble hexameric CD40-L. Such hexameric CD40-L surprisingly showed a superior cultivation and proliferation activity in the cultivation of antibody expressing cells, more specifically antibody secreting cells, such as plasmablasts.

In embodiments, the soluble CD40-L comprises a linker amino acid sequence, such as a flexible amino acid linker sequence (e.g. a serine-glycine linker, SEQ ID NO: 19 or 22) between the multimerization domain and the domain having the CD40-L activity (e.g. ectodomain or 18 kDa domain).

In embodiments, the CD40-stimulating agent (e.g. soluble CD40-L) may comprise an affinity tag (e.g, used for purification), such as an His tag, e.g. an octa-His tag. Such affinity tag may make the purification easier but is selected such that it does not negatively affect CD40-stimulating activity.

In embodiments, a CD40-stimulating agent (e.g. soluble CD40-L) may be used which comprises multiple copies (e.g. 3, 4. 5, 6, 7 or 8) of a CD40-L domain (e.g. ectodomain or 18 kDa fragment or variants thereof as described above) fused to each other in a single polypeptide. The fusion protein may comprise flexible linker sequences (e.g. serine-glycine linkers, SEQ ID NO: 19 or 22) between the copies of the CD40-L domains. Exemplary soluble CD40-L comprising linear fusions of CD40-L domains are disclosed in Merz, Christian et al. (Journal of Immunotherapy 41(9):p 385-398, November/December 2018. DOI: 10.1097/CJI.0000000000000246) and US20170327588A1.

In embodiments, the CD40-stimulating agent (e.g. soluble CD40-L) employed in the methods of the invention may comprise at least two copies of the amino acid sequence of SEQ ID NO: 1, 2, 3, 4, 5 or 6 or a variant thereof with CD40-L activity having at least 75%, at least 80%, at least 85%, at least 90%, at least 95% or at least 99% sequence identity thereto, fused directly or via a linker sequence (e.g. SEQ ID NO: 19 or 22).

In embodiments, the soluble CD40-stimulating agent (e.g. soluble CD40-L) employed in the methods of the invention may comprise at least three copies of the amino acid sequence of SEQ ID NO: 1, 2, 3, 4, 5 or 6 or a variant thereof with CD40-L activity having at least 75%, at least 80%, at least 85%, at least 90%, at least 95% or at least 99% sequence identity thereto, fused directly or via a linker sequence (e.g. SEQ ID NO: 19 or 22).

In embodiments, the soluble CD40-stimulating agent (e.g. soluble CD40-L) as used herein is recombinantely expressed, i.e. a construct that is expressed from a genetically engineered expression constructs that is not naturally occurring in nature. Molecular cloning techniques for such genetic engineering are well known in the art.

In embodiments, the CD40-stimulating agent (e.g. soluble CD40-L) as used herein may comprise any one of SEQ ID Nos: 12 to 18 or a variant thereof having at least 70%, at least 75%, at least 80%, at least 85%, at least 90%, at least 95% or at least 98% sequence identity thereto. In embodiments, the soluble CD40-L as used herein may comprise any one of SEQ ID Nos: 12 to 18 or a variant thereof having at least 85% sequence identity thereto. In embodiments, the soluble CD40-L as used herein may consist of any one of SEQ ID Nos: 12 to 18 or a variant thereof having at least 70%, at least 75%, at least 80%, at least 85%, at least 90%, at least 95% or at least 98% sequence identity thereto. In embodiments, the soluble CD40-L as used herein may consist of any one of SEQ ID Nos: 12 to 18 or a variant thereof having at least 85% sequence identity thereto. In embodiments, the soluble CD40-L as used herein may comprise any one of SEQ ID No: 12 or a variant thereof having at least 70%, at least 75%, at least 80%, at least 85%, at least 90%, at least 95% or at least 98% sequence identity thereto. In embodiments, the soluble CD40-L as used herein may comprise any one of SEQ ID No: 12 or a variant thereof having at least 85% sequence identity thereto. In embodiments, the soluble CD40-L as used herein may comprise or consist of SEQ ID Nos: 12 to 18. In embodiments, the soluble CD40-L comprises or consists of SEQ ID No: 12.

In embodiments, the CD40-stimulating agent used in the methods of the invention can be a CD40-stimulating agent (e.g. soluble CD40-L) according to the fourth aspect of the invention or any embodiments thereof.

A CD40-stimulating agent (e.g. soluble CD40-L) or a variant thereof as used herein has CD40 stimulation activity (e.g. CD40-L activity), i.e. variants lacking CD40 stimulation activity (e.g. CD40-L activity) are excluded by said term. CD40 stimulation activity means the capability to stimulate CD40 signaling, which in turn promotes cultivation and proliferation of antibody expressing cells and optionally antibody secretion in the absence of feeder cells as shown herein. In embodiments, CD40 stimulation activity (e.g. CD40-L activity) means that the capability to promote cell proliferation (in batch and/or a single cell setup) is at least as good as for the soluble CD40-L of SEQ ID NO: 12, 13, 14, 15, 16, 17 or 18, preferably of SEQ ID NO: 12 under the otherwise identical experimental conditions. Assays for assessing the capability to promote cell proliferation are described in Example 6. In brief, antibody expressing cells may be isolated by antigen panning and IgG-positive and 7-AAD negative cells may be selected and deposited as batch (i.e. 50 cells per well) into culture medium as described in Example 3. Subsequently, the cells may be cultured in batch in presence of cytokines (IL-2, IL-15, IL-21 and BAFF and the tested CD40-stimulating agent (e.g. soluble CD40-L) construct in BM1 medium. The proliferation is then assessed by determining the number of cells, e.g. after 7 days.

Alternatively or additionally, CD40-stimulating activity can be tested by stimulating naïve B cells (e.g. isolated from PBMCs via CD19-MACS from unimmunized animals) with the reagent of interest and analyzing proliferation of B cells after 4-7 days of culture via CFSE dilution in the flow cytometer. For this purpose, naïve B cells are labeled with CFSE after MACS-based purification (10 uM CFSE in RPMI medium w/o FCS and 5×106 cells/ml) for 5 minutes at 37° C. The reaction is stopped with cold medium. After a washing step, the CFSE labeled B cells are cultured in B cell medium with varying concentrations of the CD40-stimulating reagent for 4-7 days (5×10⁵ cells/well in 96-well plates). Proliferation of B cells can be tracked by analyzing CFSE labeling of living cells (7-AAD negative) in the flow cytometer. Cells that have proliferated will show diminished CFSE labeling due to dilution of the label after cell division. Naïve B cells without stimulus will not proliferate in culture, whereas naïve B cells stimulated with a CD40 stimulus will show up to 40% proliferation rates.

The soluble CD40-stimulating agent (e.g. soluble CD40-L) as used herein may be recombinantely expressed. Exemplary method and expression systems are provided in the appended Examples. The CD40-stimulating agent used in the context of the present invention is selected such that it has CD40-L activity with the antibody expressing cells of the species used. In a preferred embodiment the CD40-stimulating agent has the same species origin as the cells. In a specific embodiment, the antibody expressing cells may be rabbit cells and the CD40-stimulating agent may be any of the soluble CD40-L as described herein above or elsewhere.

The antibody expressing cell(s) (e.g. the antibody secreting cell(s) or plasmablast(s)) as used in the context of the invention may be of different origins. For example, the antibody expressing cell(s) may be rabbit, rat, murine, human, goat, hamster or sheep cells. In particular embodiments the antibody expressing cells may be rabbit or murine cells. In a particularly preferred embodiment the antibody expressing cells may be rabbit cells.

IL-2 as used in the context of the invention may be of the same or a different origin as the antibody expressing cells used. For instance, when using rabbit antibody expressing cells that IL-2 may be rabbit IL-2 or IL-2 from a different origin, provided that IL-2. As demonstrated in the appended Examples e.g. human IL-2 (UniProt: P60568) can be used for rabbit antibody expressing cells. For IL-2 from different origin, of course it needs to be confirmed that such IL-2 can stimulate the survival and proliferation of the used antibody expressing cells. This can be done by using the methods as described in the appended Examples. In a particular embodiment, the antigen-expressing cells are rabbit antigen-expressing cells and IL-2 is rabbit or human IL-2. In another embodiment, the antigen-expressing cells are rabbit antigen-expressing cells and IL-2 is human IL-2. The IL-2 is preferably recombinant.

IL-21 as used in the context of the invention may be of the same or a different origin as the antibody expressing cells used. For instance, when using rabbit antibody expressing cells that IL-21 may be rabbit IL-21 or IL-21 from a different origin, provided that IL-21. As demonstrated in the appended Examples e.g. human IL-21 (UniProt: Q9HBE4) can be used for rabbit antibody expressing cells. For IL-21 from different origin, of course it needs to be confirmed that such IL-21 can stimulate the survival and proliferation of the used antibody expressing cells. This can be done by using the methods as described in the appended Examples. In a particular embodiment, the antigen-expressing cells are rabbit antigen-expressing cells and IL-21 is rabbit or human IL-21. In another embodiment, the antigen-expressing cells are rabbit antigen-expressing cells and IL-21 is human IL-21. The IL-21 is preferably recombinant.

IL-15 as used in the context of the invention may be of the same or a different origin as the antibody expressing cells used. For instance, when using rabbit antibody expressing cells that IL-15 may be rabbit IL-15 or IL-15 from a different origin, provided that IL-15. As demonstrated in the appended Examples e.g. human IL-15 (UniProt: P40933) can be used for rabbit antibody expressing cells. For IL-15 from different origin, of course it needs to be confirmed that such IL-15 can stimulate the survival and proliferation of the used antibody expressing cells. This can be done by using the methods as described in the appended Examples. In a particular embodiment, the antigen-expressing cells are rabbit antigen-expressing cells and IL-15 is rabbit or human IL-15. In another embodiment, the antigen-expressing cells are rabbit antigen-expressing cells and IL-15 is human IL-15. The IL-15 is preferably recombinant (i.e. recombinantly expressed).

BAFF as used in the present invention is soluble BAFF, i.e. the extracellular domain of BAFF (in case of human BAFF 134 to 285) having BAFF activity. BAFF as used in the context of the invention may be of the same or a different origin as the antibody expressing cells used. For instance, when using rabbit antibody expressing cells that BAFF may be rabbit BAFF or BAFF from a different origin, provided that BAFF activity vis a vis the antibody expressing cells is provided. As demonstrated in the appended Examples e.g. human (soluble) BAFF (UniProt: Q9Y275; amino acid 134 to 285) can be used for rabbit antibody expressing cells. For BAFF from different origin, of course it needs to be confirmed that such BAFF can stimulate the survival and proliferation of the used antibody expressing cells prior to use. This can be done by using the methods as described in the appended Examples. In a particular embodiment, the antigen-expressing cells are rabbit antigen-expressing cells and BAFF is rabbit or human BAFF. In another embodiment, the antigen-expressing cells are rabbit antigen-expressing cells and BAFF is human BAFF. The BAFF is preferably recombinant.

In one embodiment all factors selected from IL-2, IL-21, IL-15 and BAFF used are from the same species origin. The species origin may be the same or different from the antibody expressing cells origin. In embodiments all factors selected from IL-2, IL-21, IL-15 and BAFF may be human (optionally with the antibody expressing cells being rabbit cells).

Concentrations for IL-2, IL-21, IL-15, BAFF and/or CD-40L used in the context of the present invention can be varied and suitable concentrations can be determined by the skilled person using the information and experiments provided herein.

Suitable concentrations of IL-2 may be 0.1 to 20000 IU/ml, in particular 10 to 10000 IU/ml, in particular 20 to 1000 IU/ml and most particularly 400 IU/ml to 900 IU/ml.

Suitable concentrations of IL-21 may be 0.1 to 150 ng/ml, in particular 1 ng/ml to 50 ng/ml and most particularly 5 ng/ml to 45 ng/ml.

Suitable concentrations of IL-15 may be 0.1 to 30 ng/ml, in particular 1 ng/ml to 15 ng/ml.

Suitable concentrations of BAFF may be 0.1 ng/ml to 300 ng/ml, in particular 1 ng/ml to 150 ng/ml and most particularly 50 ng/ml to 150 ng/ml.

Suitable concentrations of a CD40-stimulating agent will depend on the type of CD40-stimulating agent used. If the CD40-L constructs as provided herein are used, suitable concentrations may be 0.1 ng/ml to 20000 ng/ml, in particular ing/ml to 10000 ng/ml and most particularly 10 to 8000 ng/ml.

In a second aspect, the present invention relates to a method for producing an antibody specifically binding or directed to an antigen of interest, such method comprising cultivating one or more antigen binding or antigen specific antibody expressing cell(s) according to the method of the first aspect of the invention or any embodiment thereof described herein.

All definitions and embodiments disclosed herein in the context of the other aspects, such as in the context of the method according to the first aspect of the invention apply mutatis mutandis.

In embodiments, the method of the second aspect of the invention further comprises b) isolating or purifying the antibody from the culture medium and/or the antibody expressing cell(s). In some embodiments, an antibody is purified to greater than 90%, preferably 95% and most preferably 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

Methods for isolating or purifying antibodies are well known in the art. Isolating and purifying means that the antibody is separated from other components and is provided in an essentially pure form.

The isolation and/or purification of the antibody of interest may be by any conventional means such as gel filtration (size exclusion chromatography), anion exchange chromatography, cation exchange chromatography, hydrophobic interaction chromatography, high pressure liquid chromatography (HPLC), reversed phase HPLC or immunoprecipitation. These methods are well known in the art and have been generally described, e.g. in Sambrook, J & Russel, D. W. [2001](Cold Spring Harbor Laboratory, NY). The isolation and/or purification may also comprise affinity chromatography using columns with immobilized antigen of interest.

The antibody expressing cell(s) may be any antibody secreting cells obtained from peripheral blood as described in the context of the method of the first aspect of the invention herein above or herein elsewhere, provided that the cells express and/or secret the antibody specifically binding or directed to an antigen of interest. In particular embodiments, the antibody expressing cells employed in the method of the second aspect of the invention are antibody secreting cells which secret the antibody specifically binding or directed to an antigen of interest. In particular embodiments, the cells are plasmablasts (e.g. rabbit plasmablasts) secreting the antibody specifically binding or directed to an antigen of interest. The antibody expressing cells may be obtained from peripheral blood which was obtained from an animal which was previously immunized with the antigen of interest.

In a third aspect, the present invention relates to a method for producing an antibody, said method comprising:
(i) proliferating an antibody expressing cell obtained from peripheral blood according to a method of the first aspect of the invention or any embodiment thereof as disclosed herein;
(ii) determining the sequence of at least the heavy chain variable domain (VH) and the light chain variable domain (VL) of the antibody expressed by the antibody expressing cells obtained in step a);
(iii) expressing in a host cell an antibody comprising the heavy chain variable domain and the light chain variable domain encoded by the heavy chain variable domain and the light chain variable domain determined in (ii), respectively; and
(iv) isolating the expressed antibody.

The antibody expressing cell(s) may be any antibody secreting cells obtained from peripheral blood as described in the context of the method of the first aspect of the invention herein above or herein elsewhere, provided that the cells express and/or secret the antibody specifically binding or directed to an antigen of interest. In particular embodiments, the antibody expressing cells employed in the method of the second aspect of the invention are antibody secreting cells which secret the antibody specifically binding or directed to an antigen of interest. In particular embodiments, the cells are plasmablasts (e.g. rabbit plasmablasts) secreting the antibody specifically binding or directed to an antigen of interest. The antibody expressing cells may be obtained from peripheral blood which was obtained from an animal which was previously immunized with the antigen of interest. In embodiments, the peripheral blood may be obtained 4 days after immunization until 15 days after immunization or the most recent boost. In one embodiment, the peripheral blood may be obtained after 4 days until at most 9 days after immunization or the most recent boost.

Methods for the determination of the sequences of the heavy chain variable domain (VH) and the light chain variable domain (VL) may comprise
a) extracting total RNA from the antibody expressing cells;
b) performing a cDNA synthesis/reverse transcription of the extracted polyA-mRNA;
c) performing a PCR with a set of species specific primers for amplification of the VH and VL encoding sequences
d) sequencing of the amplified sequences.

Alternatively, methods for the determination of the sequences of the heavy chain variable domain (VH) and the light chain variable domain (VL) may comprise
a) extracting total RNA from the antibody expressing cells;
b) performing a cDNA synthesis/reverse transcription of the extracted polyA-mRNA;
c) cloning the amplified cDNA into a plasmid
d) sequencing of the VH and VL sequence by sequencing the cDNA cloned in the plasmid.

In embodiments, determining the sequence of VHs and VLs may be conducted as follows: The antibody expressing cells proliferated in a) are lysed for a total RNA isolation. After this a cDNA synthesis is conducted, e.g. using RT-PCR (real time-polymerase chain reaction) and an anchored oligo-dT primer which binds on poly A tail. Followed by a real time heavy chain and light chain PCR with specific primers, the heavy chain and light chain will be inserted via T4 polymerase and SLIC (sequence- and ligation-independent cloning) or PIPE (polymerase incomplete primer extension) cloning into a target plasmid (e.g. an expression plasmid). The final steps are the transformation of E. coli with the plasmids to generate biomass and a pDNA (plasmid DNA) isolation as well as a control digestion and gel separation. This plasmids are also used, in combination with specific primers, for the sequencing.

For expression in a host cell the coding sequence for an antibody comprising the sequenced VH and VL sequence may be cloned into an expression vector, which can then be transfected into a host cell. The cloning may be conducted as described in the context of sequencing above with the exception of using expression plasmids/vectors.

Suitable host cells for antibody expression include prokaryotic and eukaryotic cells (e.g. HEK or CHO cells).

Methods and strategies for determining sequences of VH and VL domains from B cells are also described in Seeber S et al. (PLoS One. 2014 Feb. 4; 9(2):e86184. doi: 10.1371/journal.pone.0086184. PMID: 24503933) and Rashidian, J. (Methods in Molecular Biology, vol 2070. Humana, New York, NY. https://doi.org/10.1007/978-1-4939-9853-1 23), which are both incorporated herein in their entirety.

The embodiments of the other aspects of the invention apply to the method of the third aspect mutatis mutandis.

In one embodiment, the antibody may be an IgG, in particular a rabbit IgG. In embodiments, the antibody may be a humanized or chimeric antibody.

In one embodiment of the invention the method may comprise culturing multiple antibody expressing cells in different cultivation vessels and selecting such antibody expressing cells that secret an antibody specifically binding to the antigen and/or epitope of interest. In other words, the method may be part of a B-cell cloning method for identifying an antibody specifically binding to a target of interest.

In a fourth aspect, the present invention relates to a CD40-stimulating agent (e.g. CD40-L), in particular to a non-cell surface presented CD40 ligand (CD40-L), in particular to a soluble CD40-L.

A CD40-stimulating agent relates to a compound (e.g. a soluble CD40-L) having CD40-stimulating activity.

CD40 stimulation activity means the capability to stimulate CD40 signaling, which in turn promotes cultivation and proliferation of antibody expressing cells and optionally antibody secretion in the absence of feeder cells as shown herein. In embodiments CD40-L activity means that the capability to promote cell proliferation (in batch and/or a single cell setup) is at least as good as for the soluble CD40-L of SEQ ID NO: 12, 13, 14, 15, 16, 17 or 18, preferably of SEQ ID NO: 12 under the otherwise identical experimental conditions. Assays for assessing the capability to promote cell proliferation are described in Example 6. In brief, antibody expressing cells may be isolated by antigen panning and IgG-positive and 7-AAD negative cells may be selected and deposited as batch (i.e. 50 cells per well) into culture medium as described in Example 3. Subsequently, the cells may be cultured in batch in presence of cytokines (IL-2, IL-15, IL-21 and BAFF and the tested soluble CD40-L construct in BM1 medium. The proliferation is then assessed by determining the number of cells, e.g. after 7 days.

Alternatively or additionally, CD40-stimulating activity can be tested by stimulating naïve B cells (e.g. isolated from PBMCs via CD19-MACS from unimmunized animals) with the reagent of interest and analyzing proliferation of B cells after 4-7 days of culture via CFSE dilution in the flow cytometer. For this purpose, naïve B cells are labeled with CFSE after MACS-based purification (1OuM CFSE in RPMI medium w/o FCS and 5×106 cells/ml) for 5 minutes at 37° C. The reaction is stopped with cold medium. After a washing step, the CFSE labeled B cells are cultured in B cell medium with varying concentrations of the CD40-stimulating reagent for 4-7 days (5×10⁵ cells/well in 96-well plates). Proliferation of B cells can be tracked by analyzing CFSE labeling of living cells (7-AAD negative) in the flow cytometer. Cells that have proliferated will show diminished CFSE labeling due to dilution of the label after cell division. Naïve B cells without stimulus will not proliferate in culture, whereas naïve B cells stimulated with a CD40 stimulus will show up to 40% proliferation rates.

A “non-cell presented CD40-stimulating agent” may be a soluble CD40-stimulating agent or a CD40-stimulating agent coated to a surface (e.g. inner surface of a culture vessel), wherein said surface is not a cell surface. A “soluble CD40 ligand” is soluble in cell culture medium and is a non-membrane anchored CD40-L protein or fragment thereof that is capable of binding to and stimulating CD40 in the antibody expressing cells, i.e. mimics the natural occurring CD40-L and CD40 interaction. A “CD40-stimulating agent coated to a surface” is a CD40-stimulating agent, such as a CD40 ligand that is attached (covalently or non-covalently) to a surface other than a cell surface. For example, a CD40-stimulated agent coated to a surface may be a CD40-stimulating agent that is coated (e.g. covalently or non-covalently) on the inner surface of the culture vessel used for cultivation of the antibody expressing cells.

The CD40-stimulating agent (e.g. soluble CD40-L) according to the fourth aspect of the invention is characterized in that it comprises a CD40-L domain. The CD40-L domain may consist of the amino acid sequence as depicted in SEQ ID NO: 1 or an amino acid sequence having at least 75%, 80%, 90%, 95% or 99% sequence identity thereto. This fragment has been shown to be sufficient for CD40 stimulation and is conserved to a sequence identity of more than 75% between rabbit and human, mouse or rat. The sequence of SEQ ID NO: 1 is a rabbit sequence. Thus, in embodiments, the soluble CD40-L as used may comprise SEQ ID NO: 1 or a sequence corresponding thereto from other species (e.g. human, mouse, rat, hamster or sheep). In embodiments, the CD40-L may comprise any one of SEQ ID Nos: 1, 2, 3, 4, 5 or 6 or a sequence having at least 75% sequence identity thereto. In embodiments, the CD40-L may comprise any one of SEQ ID Nos: 1, 2, 3, 4, 5 or 6 or a sequence having at least 80% sequence identity thereto. In embodiments, the CD40-L may comprise any one of SEQ ID Nos: 1, 2, 3, 4, 5 or 6 or a sequence having at least 85% sequence identity thereto. In embodiments, the CD40-L may comprise any one of SEQ ID Nos: 1, 2, 3, 4, 5 or 6 or a sequence having at least 90% sequence identity thereto. In embodiments, the CD40-L may comprise any one of SEQ ID Nos: 1, 2, 3, 4, 5 or 6 or a sequence having at least 95% sequence identity thereto. In embodiments, the CD40-L may comprise any one of SEQ ID Nos: 1, 2, 3, 4, 5 or 6 or a sequence having at least 99% sequence identity thereto.

In embodiments, the soluble CD40-L comprises the soluble 18 kDa-CD40-L domain, e.g., of SEQ ID NO: 1, 2, 3, 4, 5 or 6 or a sequence with a sequence identity a defined above, but no other CD40-L specific domains or sequences. As demonstrated in the appended Examples, using the 18 kDa CD40-L domain facilitated proliferation of antibody expressing cells, in particular plasmablasts, better than using the entire ectodomain (extracellular domain) of CD40-L (see SEQ ID NO: 7 for corresponding rabbit sequence). Thus, in embodiments, the soluble CD40-L as used herein does not comprises any other CD40-L ectodomain sequences other than the CD40-L 18 kDa domain (e.g. of SEQ ID NO: 1, 2, 3, 4, 5 or 6 or a sequence with a sequence identity a defined above).

In embodiments, the soluble CD40-L as used herein may comprise the amino acid sequence of SEQ ID NO: 1, 2, 3, 4, 5 or 6 or a variant thereof with CD40-L activity having at least 75%, at least 80%, at least 85%, at least 90%, at least 95% or at least 99% sequence identity thereto.

The CD40-stimulating agent according to the fourth aspect of the invention according to the fourth aspect is typically recombinantly expressed.

In embodiments, the CD40-L domain comprised in the CD40-stimulating agent of the fourth aspect has a molecular weight of about 18 kDa or 18 kDa.

The CD40-stimulating function by a CD40-L construct is facilitated by multimerization. As demonstrated in the appended Examples hexameric soluble CD40-L constructs surprisingly facilitate cultivation and proliferation of antibody expressing cells with better efficacy as trimeric soluble CD40-L constructs. Accordingly, in embodiments, the CD40-L construct is at least trimeric or more than trimeric. In an even more preferred embodiment, the CD40-L construct is at least tetrameric, more preferably at least hexameric or hexameric.

Accordingly, the CD40-stimulating agent of the invention (e.g. the soluble CD40-L) may comprise a multimerization domain, in particular an artificially introduced multimerization domain (i.e. a multimerization domain introduced by genetic engineering).

Multimerization domains leading to different stoichiometry of multimerization are known in the art. In principle, any multimerization domain resulting at least in an at least trimeric and even more preferably at least a hexameric CD40-L construct can be used. Additional degree of multimerization may occur by multimerization caused by the CD40-L domain, e.g. the 18 kDa domain itself. The degree of multimerization of a CD40-L construct can be analyzed by HPLC (e.g. using SEC-MALS) analyses. Corresponding examples of such methods are employed and described in the appended Examples. The multimerization domain in the context of the invention is selected such that the CD40-L activity of a fusion protein is maintained or improved, i.e. the activity to stimulate the CD40 receptor is not negatively affect. The person skilled in the art is well aware of multimerization domains that can be selected. Exemplary but non-limiting multimerization domains that may be comprised in the CD40-stimulating agent (e.g. soluble CD40-L) of the fourth aspect of the invention are an Fc domain (e.g. of an IgG antibody), a leucine zipper domain (LZ) and a C4b domain. In embodiments an Fc domain, in particular an IgG Fc domain, in particular a human IgG Fc domain and in particular a human IgG1 Fc domain may be used as multimerization domain.

An exemplary C4b sequence that may be used in the context of the invention is SEQ ID NO: 8 or a variant thereof having at least 80%, at least 85%, at least 90%, at least 95% or at least 98% sequence identity to SEQ ID NO: 8, said variant showing the same multimerization as SEQ ID NO: 8 if used in the CD40-L construct.

An exemplary leucine zipper sequence that may be used in the context of the invention is SEQ ID NO: 9 or a variant thereof having at least 80%, at least 85%, at least 90%, at least 95% or at least 98% sequence identity to SEQ ID NO: 9, said variant showing the same multimerization degree (e.g. trimeric) as SEQ ID NO: 9 if used in the CD40-L construct. As demonstrated in the appended Examples, this leucine zipper achieved a trimerization of the CD40-L construct.

Another exemplary leucine zipper sequence that may be used in the context of the invention is SEQ ID NO: 10 or a variant thereof having at least 80%, at least 85%, at least 90%, at least 95% or at least 98% sequence identity to SEQ ID NO: 10, said variant showing the same multimerization degree (e.g. trimeric) as SEQ ID NO: 10 if used in the CD40-L construct. As demonstrated in the appended Examples, this leucine zipper achieved a trimerization of the CD40-L construct.

Exemplary leucine zipper are also described in Burkhardt et al., Cancer Immunol Immunother. 2013 February; 62(2):347-57. doi: 10.1007/s00262-012-1331-4. Epub 2012 Aug. 25.

An exemplary IgG Fe sequence that may be used in the context of the invention is SEQ ID NO: 11 or a variant thereof having at least 80%, at least 85%, at least 90%, at least 95% or at least 98% sequence identity to SEQ ID NO: 11, said variant showing the same multimerization (e.g. hexameric) as SEQ ID NO: 11 if used in the CD40-L construct.

As demonstrated in the appended Examples using an Fc domain (e.g. of a human IgG1) results in a soluble hexameric CD40-L. Such hexameric CD40-L surprisingly showed a superior cultivation and proliferation activity in the cultivation of antibody expressing cells, more specifically antibody secreting cells, such as plasmablasts.

In embodiments, the CD40-stimulating agent of the fourth aspect (e.g. soluble CD40-L) comprises a linker amino acid sequence, such as a flexible amino acid linker sequence (e.g. a serine-glycine linker, SEQ ID NO: 19 or 22) between the multimerization domain and the domain having the CD40-L activity (e.g. ectodomain or 18 kDa domain).

In embodiments, the CD40-stimulating agent of the fourth aspect (e.g. soluble CD40-L) may comprise an affinity tag (e.g, used for purification), such as an His tag, e.g. an octa-His tag.

In embodiments, the CD40-stimulating agent of the fourth aspect (e.g. soluble CD40-L) is recombinantely expressed, i.e. a construct that is expressed from a genetically engineered expression constructs that is not naturally occurring in nature. Molecular cloning techniques for such genetic engineering are well known in the art.

In embodiments, the CD40-stimulating agent of the fourth aspect (e.g. soluble CD40-L) may comprise any one of SEQ ID Nos: 12 to 18 or a variant thereof having at least 70%, at least 75%, at least 80%, at least 85%, at least 90%, at least 95% or at least 98% sequence identity thereto. In embodiments, the soluble CD40-L as used herein may comprise any one of SEQ ID Nos: 12 to 18 or a variant thereof having at least 85% sequence identity thereto. In embodiments, the soluble CD40-L as used herein may consist of any one of SEQ ID Nos: 12 to 18 or a variant thereof having at least 70%, at least 75%, at least 80%, at least 85%, at least 90%, at least 95% or at least 98% sequence identity thereto. In embodiments, the soluble CD40-L as used herein may consist of any one of SEQ ID Nos: 12 to 18 or a variant thereof having at least 85% sequence identity thereto. In embodiments, the soluble CD40-L as used herein may comprise any one of SEQ ID No: 12 or a variant thereof having at least 70%, at least 75%, at least 80%, at least 85%, at least 90%, at least 95% or at least 98% sequence identity thereto. In embodiments, the soluble CD40-L as used herein may comprise any one of SEQ ID No: 12 or a variant thereof having at least 85% sequence identity thereto. In embodiments, the soluble CD40-L as used herein may comprise or consist of SEQ ID Nos: 12 to 18. In embodiments, the soluble CD40-L comprises or consists of SEQ ID No: 12.

A CD40-stimulating agent, such as a CD40-L (e.g. soluble CD40-L) or a variant thereof as provided according to the fourth aspect of the invention has CD40 stimulation activity, i.e. variants lacking CD40-L activity are excluded by said term. CD40 stimulation activity means the capability to stimulate CD40 signaling, which in turn promotes cultivation and proliferation of antibody expressing cells and optionally antibody secretion in the absence of feeder cells as shown herein. In embodiments CD40-L activity means that the capability to promote cell proliferation (in batch and/or a single cell setup) is at least as good as for the soluble CD40-L of SEQ ID NO: 12, 13, 14, 15, 16, 17 or 18, preferably of SEQ ID NO: 12 under the otherwise identical experimental conditions. Assays for assessing the capability to promote cell proliferation are described in Example 6. In brief, antibody expressing cells may be isolated by antigen panning and IgG-positive and 7-AAD negative cells may be selected and deposited as batch (i.e. 50 cells per well) into culture medium as described in Example 3. Subsequently, the cells may be cultured in batch in presence of cytokines (IL-2, IL-15, IL-21 and BAFF and the tested soluble CD40-L construct in BM1 medium. The proliferation is then assessed by determining the number of cells, e.g. after 7 days. Alternatively or additionally, CD40-stimulating activity can be tested by stimulating naïve B cells (e.g. isolated from PBMCs via CD19-MACS from unimmunized animals) with the reagent of interest and analyzing proliferation of B cells after 4-7 days of culture via CFSE dilution in the flow cytometer. For this purpose, naïve B cells are labeled with CFSE after MACS-based purification (10 uM CFSE in RPMI medium w/o FCS and 5×106 cells/ml) for 5 minutes at 37° C. The reaction is stopped with cold medium. After a washing step, the CFSE labeled B cells are cultured in B cell medium with varying concentrations of the CD40-stimulating reagent for 4-7 days (5×10⁵ cells/well in 96-well plates). Proliferation of B cells can be tracked by analyzing CFSE labeling of living cells (7-AAD negative) in the flow cytometer. Cells that have proliferated will show diminished CFSE labeling due to dilution of the label after cell division. Naïve B cells without stimulus will not proliferate in culture, whereas naïve B cells stimulated with a CD40 stimulus will show up to 40% proliferation rates.

The CD40-stimulating agent, in particular (soluble) CD40-L, of the fourth aspect of the present disclosure is typically recombinantely expressed.

Exemplary method and expression systems are provided in the appended Examples. As described in the appended examples expression using eukaryotic expression systems such as HEK 293T cells may be used. Exemplary methods for purification of CD 40 stimulating agents of the invention are also described in the appended Examples.

In a fifth aspect, the present invention relates to the use of the CD40-stimulating agent (e.g. soluble CD40-L) according to the fourth aspect of the invention for cultivating and/or proliferating one or more antibody expressing cells obtained from peripheral blood ex vivo (e.g., plasmablasts) in the absence of feeder cells.

The embodiments and definitions provided herein above in the context of the first, second, third and fourth aspects apply mutatis mutandis.

In embodiments the use involves any of the method steps as defined in the context of the method of the first aspects and its embodiments.

In specific embodiments, the cultivation and/or proliferation of the use according to the fifth aspect is performed in presence of IL-2 and IL-21. Optionally, cultivation and/or proliferation may be performed in further presence of IL-15 and/or BAFF.

Preferred concentration ranges of these cytokines are described in the context of the method of the first aspect and in the appended Examples.

In embodiments, the cultivation and/or proliferation of the one or more antibody expressing cells is performed in a single cell format.

In embodiments, the one or more antibody expressing cell(s) obtained from peripheral blood is/are rabbit cells.

In embodiments, the one or more antibody expressing cell(s) obtained from peripheral blood are antibody secreting cells, in particular plasmablasts. Methods for obtaining such cells are described in the context of the method of the first aspect herein above and in the appended Examples.

In embodiments, the antibody expressing cells are primary.

In a sixth embodiment, the present invention relates to the use of a CD40-stimulating agent (e.g. non-cell surface bound CD40-L) according to the fourth aspect of the invention or any embodiments thereof for B cell cloning. The use preferably comprises cultivating the primary B cells obtained from peripheral blood in the presence of the CD40-ligand (CD40-L) according to the fourth aspect of the invention or any embodiments thereof. In embodiments, the cultivation is performed in further presence of IL-2 and IL-21. Optionally, IL-15 or (soluble) BAFF may be present during cultivation. Preferably the cultivation is conducted in the absence of feeder cells. Preferably, the cultivation is performed in the absence of conditioned cell supernatant, such as, for example, a thymocyte supernatant (TSN).

The embodiments and definitions disclosed in the context of any of the preceding aspects apply mutatis mutandis.

B cell cloning technologies are methods for screening monoclonal antibodies from immunized animals, involving isolating primary B cells, screening B cells expressing antibodies of interest and obtaining said antibodies therefrom or determine the sequences of the expressed VH and VL domains. Subsequently, antibodies comprising the sequenced VH and VL domains can be expressed recombinantely, e.g. using CHO or HEK cells. Alternatively, in principle instead of sequencing also hybridoma cells may be generated from the isolated B cells expressing antibodies with the desired characteristics.

In a seventh aspect, the present invention provides a cell culture medium, said cell culture medium comprising a non-cell surface bound CD40-stimulating agent (e.g. soluble CD40-L) of the fourth aspect of the invention or any embodiments thereof.

In embodiments, the cell culture medium further comprises IL-2 and/or IL-21. In embodiments the cell culture medium comprises IL-15. In embodiments, the cell culture medium comprises soluble BAFF. In embodiments, the cell culture medium comprises IL-2, IL-21 and IL-15. In embodiments the cell culture medium comprises IL-2, IL-21 and soluble BAFF. In embodiments, the cell culture medium comprises IL-2, IL-21, IL-15 and soluble BAFF.

In embodiments the cell culture medium does not comprise one or more selected from: ICOS, 4-1BB, PMA and IL-4. In certain embodiments, the cell culture medium does not comprise ICOS, 4-1BB, PMA and IL-4.

The cell culture medium may otherwise be configured as a B cell culture medium. In certain embodiments, the cell culture medium may be based on RPMI medium, such as RPMI 1640 medium (e.g. Gibco, 31870). In embodiments, the cell culture medium may be based on eRDF medium (Murakami H, 1989, Advances in Biotechnological Processes, Vol. 11, Monoclonal Antibodies: Production and Application. Alan R. Liss, New York, pp. 107-141). In certain embodiments, the medium may comprise one or more or all of glutamine, pyruvate, B-Mercapthoethanol and a buffer (e.g. Hepes). In embodiments, the cell culture medium may comprise fetal calf serum (FCS), e.g. 10 or less or 5%. In certain embodiments, the medium may comprise one or more or all of fetal calf serum, glutamine and Mercaptoptoethanol. In embodiments, the cell culture medium may comprise Staphyalococcus aureus cells (SAC).

Exemplary cell culture media components are the components of the BM1 and BM2 medium as described in the appended Examples.

As demonstrated in the appended Examples, the cell culture medium of the invention can promote in vivo survival and proliferation of primary antibody expressing cells obtained from peripheral blood (e.g. of a rabbit) in bulk or in a single cell format, even in the absence of feeder cells and conditioned cell supernatant, such as, for example, a thymocyte supernatant (TSN).

Accordingly, the cell culture medium of the invention is a medium for culturing and/or proliferating antibody expressing cells (e.g. primary antibody expressing cells; e.g. obtained from rabbit), in particular plasmablasts (e.g. primary plasmablasts, e.g. obtained from rabbit) ex vivo.

The present invention also relates to the following items:
Item 1: An in vitro method for cultivating one or more antibody expressing cell(s) obtained from peripheral blood, said method comprising:
cultivating the one or more antibody expressing cell(s) in the presence of IL-2, IL-21 and a non-cell surface presented CD40-stimulating agent,
wherein said cultivating is performed in the absence of feeder cells.
Item 2: The method of item 1, wherein said cultivating of the one or more antibody expressing cell(s) is performed in the presence of IL-15 and/or BAFF.
Item 3: The method of item 1 or 2, wherein said cultivating of the one or more antibody expressing cell(s) is performed in the absence of conditioned cell supernatant, in particular a thymocyte supernatant (TSN).
Item 4: The method of any one of items 1 to 3, wherein said cultivating of the one or more antibody expressing cell(s) is performed in the absence of one or more selected from: ICOS, 4-1BB, PMA and IL-4.
Item 5: The method of any one of items 1 to 4, wherein the one or more antibody expressing cell(s) is/are primary.
Item 6: The method of any one of items 1 to 5, wherein the one or more antibody expressing cell(s) are 100 or less antibody expressing cell(s), preferably 75 or less antibody expressing cell(s), even more preferably 50 or less antibody expressing cell(s) and even more preferably 20 or less antibody expressing cell(s).
Item 7: The method of any one of items 1 to 6, wherein the one or more antibody expressing cell(s) is one antibody expressing cell, and wherein said cultivating is performed in a single cell format.
Item 8: The method of any one of items 1 to 7, wherein the antibody expressing cells express and/or secret IgG, IgM or IgA.
Item 9: The method of any one of items 1 to 8, wherein said method further comprises isolating the one or more antibody expressing cell(s) from an obtained peripheral blood sample.
Item 10: The method of item 9, wherein isolating the one or more antibody expressing cell(s) from peripheral blood comprises isolating PBMCs from the obtained peripheral blood sample and isolating the one or more antibody expressing cell(s) from the isolated PBMCs.
Item 11: The method of item 9 or 10, wherein the one or more antibody expressing cell(s) is/are one or more antigen specific antibody expressing cell(s) and the isolating the one or more antibody expressing cell(s) further comprises isolating cells expressing one or more antibodies binding to an antigen of interest.
Item 12: The method of item 10 or 11, wherein said isolation of one or more antibody expressing cells from peripheral blood comprises isolating cells expressing a membrane bound antibody, e.g. a membrane bound IgG, IgA or IgM.
Item 13: The method of any one of items 10 to 12, wherein said isolation of one or more antibody expressing cells from peripheral blood comprises
i) isolating PBMCs from peripheral blood;
ii) isolating from said PBMCs cells that are: a) IgG^{int} and b) have an intermediate cell size (FSC^{int}) preferably using flow cytometry; and
iii) optionally isolating from the PBMCs antigen specific antibody expressing cells binding to an antigen of interest at their cell surface.
Item 14: The method of any one of items 1 to 13, wherein the one or more antibody expressing cell(s) is/are one or more antibody secreting cells.
Item 15: The method of any one of item 1 to 14, wherein the one or more antibody expressing cell(s) comprise or consist of one or more plasmablasts.
Item 16: The method of any one of item 1 to 15, wherein the one or more antibody expressing cell(s) comprise or consist of one or more IgG expressing cell(s) and wherein the one or more IgG expressing cell(s) have an intermediate IgG expression (IgG^{int}) and an intermediate cell size (FSC^{int}), preferably as assessed by flow cytometry.
Item 17: The method of item 16, wherein the one or more IgG expressing cell(s) express antibodies directed to the same antigen of interest.
Item 18: The method of any one of item 1 to 17, wherein the one or more antibody expressing cell(s) are CD19-positive and/or CD43-positive.
Item 19: The method of any one of items 1 to 18, wherein the one or more antibody expressing cell(s) are obtainable or obtained by
i) isolating PBMCs from peripheral blood;
ii) isolating from said PBMCs cells that are: a) IgG^{int} and b) have an intermediate cell size (FSC^{int}), preferably using flow cytometry; and
iii) optionally isolating from the PBMCs, cells binding to an antigen of interest at their cell surface.
Item 20: The method of any one of items 1 to 19, wherein the one or more antibody expressing cells are rabbit, rat, murine, human, goat, hamster or sheep cell(s).
Item 21: The method of any one of items 1 to 19, wherein the antibody expressing cells are rabbit cell(s).
Item 22: The method of any one of items 1 to 21, wherein the non-cell surface presented CD40-stimulating agent is a soluble CD40-stimulating agent, in particular a soluble CD40-ligand (CD40-L).
Item 23: The method of any one of items 1 to 22, wherein the non-cell surface presented CD40-stimulating agent comprises a soluble 18 kDa-CD40-L domain.
Item 24: The method of any one of items 1 to 23, wherein the non-cell surface presented CD40-stimulating agent comprises the amino acid sequence of SEQ ID NO: 1, 2, 3, 4, 5 or 6 or a variant thereof with CD40-L activity having at least 75%, at least 80%, at least 85%, at least 90%, at least 95% or at least 99% sequence identity thereto.
Item 25: The method of any one of items 1 to 21, wherein the non-cell surface presented CD40-stimulating agent is a soluble CD40-ligand (CD40-L).
Item 26: The method of item 25, wherein the soluble CD40-L is at least trimeric, in particular at least hexameric, in particular hexameric.
Item 27: The method of item 25 or 26, wherein the CD40-L comprises a multimerization domain.
Item 28: The method of item 27, wherein the multimerization domain is selected from the group consisting of Fc domain, leucine zipper, and C4b.
Item 29: The method of item 27, wherein the multimerization domain is an Fc domain, preferably the Fc domain from human IgG, preferably the Fc domain of human IgG1, IgG2, IgG3 or IgG4; murine IgG1, IgG2a, IgG2b, or IgG3; rabbit IgG.
Item 30: The method of item 27, wherein the multimerization domain is a human IgG1 Fc domain, in embodiments the Fc domain having the amino acid sequence of SEQ ID: 11 or a variant thereof capable of multimerization and having a sequence identity to SEQ ID NO: 11 of at least 80%.
Item 31: The method of item 27, wherein the multimerization domain comprises an amino acid sequence selected from SEQ ID NOs: 11, 8, 9, 10, or a variant of any one of the aforementioned having at least 80% sequence identity and being capable of multimerizing the soluble CD40-L.
Item 32: The method of any one of items 1 to 31, wherein the one or more antibody expressing cell(s) are rabbit cell(s).
Item 33: The method of item 32, wherein the IL-2, IL-21 and optionally the IL-15 and/or BAFF is/are each independently rabbit or human protein(s), preferably recombinantly expressed protein(s).
Item 34: The method of item 32 or 33, wherein the CD40-stimulating agent is a rabbit CD40-stimulating agent.
Item 35: The method of any one of items 1 to 34, wherein IL-15 is present at a concentration of 0.1 to 30 ng/ml, in particular 1 ng/ml to 15 ng/ml.
Item 36: The method of any one of items 1 to 35, wherein IL-21 is present at a concentration of 0.1 to 150 ng/ml, in particular 1 ng/ml to 50 ng/ml and most particularly 5 ng/ml to 45 ng/ml.
Item 37: The method of any one of items 1 to 36, wherein BAFF is present at a concentration of 0.1 ng/ml to 300 ng/ml, in particular 1 ng/ml to 150 ng/ml and most particularly 50 ng/ml to 150 ng/ml.
Item 38: The method of any one of items 1 to 37, wherein IL2 is present at 0.1 to 20000 IU/ml, in particular 10 to 10000 IU/ml, in particular 20 to 1000 IU/ml and most particularly 400 IU/ml to 900 IU/ml Item 39: The method of any one of items 1 to 38, wherein the CD40-stimulating agent is present at a concentration of 0.1 ng/ml to 20000 ng/ml, in particular 1 ng/ml to 10000 ng/ml and most particularly 10 to 8000 ng/ml.
Item 40: A method for producing an antibody (e.g. monoclonal antibody) directed to an antigen of interest comprising:
(i) cultivating one or more antibody expressing cell(s) obtained from peripheral blood according to the method as defined in any one of items 1 to 39, optionally wherein all said one or more antibody expressing cell(s) express an antibody directed to the same antigen of interest; and
(ii) isolating the antibody from the culture medium and/or the antibody expressing cell(s).
Item 41: The method of item 40, wherein the method further comprises immortalizing the one or more antibody expressing cell(s).
Item 42: A method for producing an antibody (e.g. monoclonal antibody) comprising:
(i) proliferating an antibody expressing cell obtained from peripheral blood using a cultivation method of any one of items 1 to 39;
(ii) determining the sequence of at least the heavy chain variable domain (VH) and the light chain variable domain (VL) of the antibody expressed by the antibody expressing cells obtained by the proliferation in step a);
(iii) expressing in a host cell an antibody comprising the heavy chain variable domain and the light chain variable domain encoded by the heavy chain variable domain sequence and the light chain variable domain sequence determined in (ii), respectively; and
(iv) isolating the expressed antibody.
Item 43: The method of any one of items 40 to 42, wherein the antibody expressing cell is an antibody expressing cell as defined in any one of items 8 to 21.
Item 44: The method of any one of items 40 to 43, wherein the antibody is an IgG.
Item 45: The method of any one of items 40 to 44, wherein the antibody is a rabbit IgG.
Item 46: The method of any one of items 40 to 45, wherein the antibody is specifically binding to an antigen and/or epitope of interest, wherein the method comprises selecting the antibody expressing cell based on the fact that expresses an antibody specifically binding to the antigen and/or epitope of interest.
Item 47: A non-cell surface bound CD40-stimulating agent comprising an CD40-L domain comprising or consisting of the amino acid sequence of SEQ ID NO: 1, 2, 3, 4, 5 or 6 or a variant thereof with CD40-L activity having at least 75%, at least 80%, at least 85%, at least 90%, at least 95% or at least 99% sequence identity thereto.
Item 48: The CD40-stimulating agent of item 47, wherein the CD40-stimulating agent is at least trimeric, in particular at least hexameric, in particular hexameric.
Item 49: The CD40-stimulating agent of item 47 o 48 comprising a multimerization domain.
Item 50: The CD40-stimulating agent of item 49, wherein the multimerization domain is selected from the group consisting of Fc domain, leucine zipper, and C4b.
Item 51: The CD40-stimulating agent of item 49, wherein the multimerization domain is an Fc domain, preferably the Fc domain from human IgG, preferably the Fc domain of human IgG1, IgG2, IgG3 or IgG4; murine IgG1, IgG2a, IgG2b, or IgG3; rabbit IgG.
Item 52: The CD40-stimulating agent of item 49, wherein the multimerization domain is a human IgG1 Fc domain, in embodiments the Fc domain having the amino acid sequence of SEQ ID: 11 or a variant thereof capable of multimerization and having a sequence identity to SEQ ID NO: 11 of at least 80%.
Item 53: The CD40-stimulating agent of item 49, wherein the multimerization domain comprises an amino acid sequence selected from SEQ ID NOs: 11, 8, 9, 10, or a variant of any one of the aforementioned having at least 80% sequence identity and being capable of multimerizing the soluble CD40-L.
Item 54: The CD40-stimulating agent of any one of items 49 to 53, wherein the CD40-stimulating agent comprises a linker, such as a linker amino acid sequence (e.g. SEQ ID NO: 19 or 22) between the CD40-L domain and the multimerization domain.
Item 55: The CD40-stimulating agent of any one of items 47 to 54, wherein the CD40-stimulating agent is a CD40-L construct, in particular is a soluble CD40-L construct.
Item 56: Use of the non-cell surface bound CD40-stimulating agent as defined in any of items any one of items 47 to 55 for cultivating and/or proliferating one or more antibody expressing cells obtained from peripheral blood ex vivo in the absence of feeder cells.
Item 57: The use of item 56, wherein the cultivation and/or proliferation is performed in presence of IL-2 and 11-21.
Item 58: The use of item 56 or 57, wherein the cultivation and/or proliferation is performed in presence of IL-15 and/or BAFF.
Item 59: The use of any one of items 56 to 58, wherein the one or more antibody expressing cells is performed in a single cell format.
Item 60: The use of any one of items 56 to 58, wherein the one or more antibody expressing cells are as defined in any one of items 8 to 21.
Item 61: Use of the non-cell surface bound CD40-stimulating agent as defined in any one of items 47 to 55 for B cell cloning technologies, preferably in the absence of feeder cells.
Item 62: A cell culture medium comprising a non-cell surface bound CD40-stimulating agent as defined in any of items 47 to 55, IL-2 and IL-21.
Item 63: The cell culture medium of item 62 further comprising IL-15.
Item 64: The cell culture medium of item 62 or 63 further comprising BAFF (soluble form).
Item 65: The cell culture medium of any one of items 62 to 64 not comprising one or more selected from: ICOS, 4-1BB, PMA and IL-4.
Item 66: The cell culture medium of any one of items 62 to 65, wherein the cell culture medium is a medium for culturing antibody expressing cells (e.g. primary antibody expressing cells; e.g. obtained from rabbit), in particular plasmablasts (e.g. primary plasmablasts, e.g. obtained from rabbit).

The following definitions apply to all aspects, embodiments and items of the present invention disclosed herein.

In general, terms used herein are to be given their ordinary and customary meaning to a person of ordinary skill in the art and, unless indicated otherwise, are not to be limited to a special or customized meaning.

As used herein, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” in an embodiment refer to “comprising one or more”, i.e. are equivalent to “comprising at least one”. In accordance, expressions relating to one item of a plurality, unless otherwise indicated, in an embodiment relate to at least one such item, in a further embodiment a plurality thereof, thus, e.g. identifying “a cell” relates to identifying at least one cell, in an embodiment to identifying a multitude of cells.

In accordance, the terms “one or more” or “at least one”, as used herein, means that one or more of the items referred to following the term may be used or be present. For example, if the term indicates that one or more cell is to be cultivated, this may be understood as cultivating one or more than one cells, i.e. two, three, four, five or any other number. Depending on the item the term refers to, the skilled person understands as to what upper limit the term may refer, if any.

As used in this specification and the appended claims, the singular forms “a”, “an”, and “the” include plural referents, unless the content clearly dictates otherwise.

Concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “150 mg to 600 mg” should be interpreted to include not only the explicitly recited values of 150 mg to 600 mg, but to also include individual values and sub-ranges within the indicated range. Thus, included in this numerical range are individual values such as 150, 160, 170, 180, 190, . . . 580, 590, 600 mg and sub-ranges such as from 150 to 200, 150 to 250, 250 to 300, 350 to 600, etc. This same principle applies to ranges reciting only one numerical value. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

As used herein, the term “variant” is to be understood as a polypeptide or polynucleotide which differs in comparison to the polypeptide or polynucleotide from which it is derived by one or more changes in its length or sequence. The polypeptide or polynucleotide from which a polypeptide or polynucleotide variant is derived is also known as the parent polypeptide or polynucleotide. The term “variant” comprises “fragments” or “derivatives” of the parent molecule. Typically, “fragments” are smaller in length or size than the parent molecule, whilst “derivatives” exhibit one or more differences in their sequence in comparison to the parent molecule. Also encompassed are modified molecules such as but not limited to post-translationally modified proteins (e.g. glycosylated, biotinylated, phosphorylated, ubiquitinated, palmitoylated, or proteolytically cleaved proteins) and modified nucleic acids such as methylated DNA. Also mixtures of different molecules such as but not limited to RNA-DNA hybrids, are encompassed by the term “variant”. Typically, a variant is constructed artificially, preferably by gene-technological means, whilst the parent protein or polynucleotide is a wild-type protein or polynucleotide, or a consensus sequence thereof. However, also naturally occurring variants are to be understood to be encompassed by the term “variant” as used herein. Further, the variants usable in the present invention may also be derived from homologs, orthologs, or paralogs of the parent molecule or from artificially constructed variant, provided that the variant exhibits at least one biological activity of the parent molecule, i.e. is functionally active.

The term “in vitro” is used to indicate that a process or method is performed outside a living organism and preferably on body fluids, isolated tissues, organs or cells. An in vitro method may also be referred to an ex vivo method herein.

The terms “cultivating” or “culturing” as used herein relates to promoting survival of cells in a cell culture setting. This term includes maintaining cell numbers but in particular also proliferation (i.e. increasing cell numbers).

“Peripheral blood mononuclear cells (PBMCs)” are any peripheral blood cells having a round nucleus. These cells consist of lymphocytes (T cells, B cells, NK cells) and monocytes, whereas erythrocytes and platelets have no nuclei, and granulocytes (neutrophils, basophils, and eosinophils) have multi-lobed nuclei. Accordingly, PBMCs essentially consist of lymphocytes and monocytes; i.e. are lymphocytes and monocytes enriched from previously obtained peripheral blood. As will be appreciate by a skilled person, PBMCs are substantially free of other cells but may comprise a minor amount of other cells as the methods used for isolating PBMCs will hardly lead to a 100% purity. In embodiments, PBMCs consist to at least 80%, preferably at least 85%, even more preferably 90%, even more preferably 95% and most preferably at least 99% of mononuclear peripheral blood cells (i.e. lymphocytes and monocytes).

“Peripheral blood” is the blood circulating throughout the body. The cellular components that could be isolated from human peripheral blood include erythrocytes (red blood cells), leukocytes (white blood cells), and thrombocytes (platelets).

The term “antibody expressing cells” obtained from peripheral blood, as used herein, relates to cells obtained from peripheral blood which express at least at its cell surface an antibody as B cell receptor (e.g. IgG, IgM or IgA, in particular IgG). Antibody expressing cells may be B cells. Antibody expressing cells may further secret antibody (e.g. IgG, IgM or IgA, in particular IgG), i.e. may be antibody-secreting cells. In a particularly preferred embodiment, herein antibody expressing cells are plasmablasts.

The term “antibody secreting cells” as used herein relates to cells secreting antibody (e.g. IgG, IgM or IgA, in particular IgG). In specific embodiments, antibody secreting cells still express to some extent the corresponding B cell receptor at their surface, i.e. can be stained for the respective antibody.

“Plasmablasts” have an intermediate staining for IgG (IgG^{int}) and an intermediate cell size (FSCi^{int}) in PMCs as analyzed by the flow cytometer and can be selected accordingly. Plasmaplasts distinguish from memory B cells in that they are lower in IgG expression and bigger in size (FSC). Plasmablasts secret antibodies. Plasmablasts are known to express CD19 and CD43 on their cell surface (in particular in rabbits).

Cultivation of one or more cells “in the presence of” means that the subsequently substances are present in the culture medium. Exemplary basic cell culture mediums that can be used are provided herein and specific examples thereof are disclosed in the appended Examples.

A “non-cell presented CD40-stimulating agent” may be soluble CD40-stimulating agent or a CD40-stimulating agent coated to a surface (e.g. inner surface of a culture vessel), wherein said surface is not a cell surface.

“Soluble CD40-stimulating agent” means that the CD40-stimulating agent is not presented on a cell surface or an artificial surface and is soluble in the culture medium used.

A “CD40-stimulating agent coated to a surface” is a CD40-stimulating agent, such as a CD40 ligand that is attached (covalently or non-covalently) to a surface other than a cell surface. For example, a CD40-stimulated agent coated to a surface may be a CD40-stimulating agent that is coated on the inner surface of the culture vessel used for cultivation of the antibody expressing cells.

A “CD40 ligand (CD40-L)” as used herein relates to a molecule having CD40-L activity.

A “CD40 ligand domain” as used herein is a domain derived from a full length CD40-L, wherein the domain has CD40-L activity. Exemplary CD40-L domains are the ectodomain (extracellular domain) of CD40-L and the 18 kda CD40L domain as described herein.

“Feeder cells”, as used herein are cells that support the cultivation and proliferation of antibody expressing cells (e.g. antibody secreting cells, in particular plasmablasts) ex vivo. Without being bound by theory, feeder cells can promote survival and/or proliferation of antibody expressing cells by cell-cell interactions and/or the production of cytokines and potentially other factors.

As used herein, the term “% sequence identity” in connection with amino acid sequences of polypeptides/peptides and/or nucleic acid sequences or nucleic acid molecules describes the number of matches of identical amino acid or nucleic acid residues of two or more aligned sequences as compared to the number of residues making up the overall length of the compared sequences (or the overall compared portions thereof). Using an alignment of two or more sequences or subsequences, the percentage of residues that are the same may be determined when the (sub)sequences are compared and aligned for maximum correspondence over a window of comparison, or over a designated region as measured using a sequence comparison algorithm as known in the art, or when manually aligned and visually inspected. Non-limiting examples of algorithms for use in determining sequence identity include, for example, those based on the NCBI BLAST algorithm (Altschul et al., Nucleic Acids Res 25(1997), 3389-3402), CLUSTALW computer program (Thompson, Nucl. Acids Res. 2(1994), 4673-4680) or FASTA (Pearson and Lipman, Proc. Natl. Acad. Sci., 85(1988), 2444). Although the FASTA algorithm typically does not consider internal non-matching deletions or additions in sequences, i.e. gaps, in its calculation, this can be corrected manually to avoid an overestimation of the % sequence identity. CLUSTALW, however, does take sequence gaps into account in its identity calculations. Also available are the BLAST and BLAST 2.0 algorithms (Altschul et al., Nucl Acids Res., 25(1977), 3389).

The term “antibody” herein is used in the broadest sense and encompasses various antibody structures, including but not limited to monoclonal antibodies, multispecific antibodies (e.g., bispecific antibodies), chimeric antibodies and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The term “chimeric” antibody refers to an antibody in which a portion of the heavy and/or light chain is derived from a particular source or species, while the remainder of the heavy and/or light chain is derived from a different source or species.

The “class” or “type” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1 and IgA2. The heavy chain constant domains that correspond to the different classes of immunoglobulins are called α, δ, ε, γ, and μ, respectively.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In embodiments, monoclonal antibodies may be recombinantly expressed, e.g. using eukaryotic expression systems.

The term “specifically binding” and grammatical equivalents thereof denote that the antibody binds to its target with a dissociation constant (K_{D}) of 10⁻⁷ M or less, in one embodiment of from 10⁻⁸ M to 10⁻¹³ M, in a further embodiment of from 10-9 M to 10⁻¹³ M. The term is further used to indicate that the antibody does not specifically bind to other biomolecules present, i.e. it binds to other biomolecules with a dissociation constant (K_{D}) of 10⁻⁶ M or more, in one embodiment of from 10⁻⁶ M to 1 M.

The term “host cell” refers to a cell into which a nucleic acid, e.g. encoding a heterologous polypeptide, can be or is transfected. The term “host cell” includes both prokaryotic cells, which are used for propagation of plasmids, and eukaryotic cells, which are used for the expression of a nucleic acid and production of the encoded polypeptide. In one embodiment, the eukaryotic cells are mammalian cells. In one embodiment the mammalian cell is a CHO cell, optionally a CHO KT cell (ATCC CCE-61 or DSM ACC 110), or a CHO DG44 cell (also known as CHO-DHFR[-], DSM ACC 126), or a CHO XE99 cell, a CHO-T cell (see e.g. Morgan, D., et al., Biochemistry 26 (1987) 2959-2963), or a CHO-S cell, or a Super-CHO cell (Pak, S. C. O., et al. Cytotechnol. 22 (1996) 139-146). If these cells are not adapted to growth in serum-free medium or in suspension an adaptation prior to the use in the current method is to be performed.

Herein, the elements of the present invention are described. These elements are listed as aspects with specific embodiments, however, it should be understood that they may be combined in any manner and in any number to create additional aspects and embodiments. In particular, embodiments disclosed in context of one aspect apply mutatis mutandis to the other aspects. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

All references cited in this specification are herewith incorporated by reference with respect to their entire disclosure content and the disclosure content specifically mentioned in this specification.

Sequences

SEQ ID NO: 1-rabbit 18 kDa CD40-L domain capable of stimulating CD40
(Uniprot: G1SKP7, amino acids 108-261)
EINFEMQKGDQDPQIAAHLISEASSKSSSVLQWAKKGYYTMSNTLVTLENG
KQLKVKRQGFYYIYAQVTFCSNQEPSSQAPFIASLCLKSSGGSERILLRAAN
ARSSSKTCEQQSIHLGGVFELQADASVFVNVTDASQVNHGTGFTSFGLLKL
SEQ ID NO: 2-human 18 kDa CD40-L domain capable of stimulating CD40
(Uniprot: P29965, amino acids 108-261):
ENSFEMQKGDQNPQIAAHVISEASSKTTSVLQWAEKGYYTMSNNLVTLEN
GKQLTVKRQGLYYIYAQVTFCSNREASSQAPFIASLCLKSPGRFERILLRAA
NTHSSAKPCGQQSIHLGGVFELQPGASVFVNVTDPSQVSHGTGFTSFGLLKL
SEQ ID NO: 3-rat 18 kDa CD40-L domain capable of stimulating CD40 (Uniprot:
Q9Z2V2, amino acids 108-260):
EKSFEMQRGDEDPQIAAHVVSEANSNAASVLQWAKKGYYTMKSNLVVLE
NGRQLTVKREGLYYVYTQVTFCSNREPLSQRPFIVSLWLKPSSGSERILLRA
ANTHSSSKLCEQQSIHLGGVFELQAGASVFVNVTEASQVIHGIGFSSIGLLKL
SEQ ID NO: 4-mouse 18 kDa CD40-L domain capable of stimulating CD40
(Uniprot: P27548, amino acids 108-260):
ENSFEMQRGDEDPQIAAHVVSEANSNAASVLQWAKKGYYTMKSNLVMLE
NGKQLTVKREGLYYVYTQVTFCSNREPSSQRPFIVGLWLKPSSGSERILLK
AANTHSSSQLCEQQSVHLGGVFELQAGASVFVNVTEASQVIHRVGFSSFGLLKL
SEQ ID NO: 5-sheep 18 kDa CD40-L domain capable of stimulating CD40
(Uniprot: Q4TVR2, amino acids 108-261):
EKNFEMHKGDQEPQIAAHVISEASSKTTSVLQWAPKGYYTLSSNLVTLENG
KQLAVKRQGFYYIYTQVTFCSNREALNQAPFIASLCLKSPSGSERILLRAAN
THSSSKPCGQQSIHLGGVFELQQGASVFVNVTDPSQVSHGTGFTSFGLLKL
SEQ ID NO: 6-Chinese hamster 18 kDa CD40-L domain capable of stimulating
CD40 (Uniprot: A0A3L7H1M2, amino acids 108-260):
IKLNTEEKKENSYEMQKGDEDPQIAVHVVSEANSKTASVLQWAKKGYYT
MKNNLVMLESGKQLTIKRQGLYYVYTQVTFCSNQGPSSKDPFLVSLCLKS
TSGSERILLRAANTHSSSKPCGQQSVHLGGVFELQEDSSLFVNVTDASQVIH
GIGFTSFGLLKL
SEQ ID NO: 7-rabbit CD40-L domain extracellular domain (ectodomain); UniProt:
G1SKP7, amino acids 47-261
HRRLDKIEDERNLHEDFVFMKTIQRCNKGEGSLSLLNCKEIRSQFEGFVK
DIMLNKEEPKKEINFEMQKGDQDPQIAAHLISEASSKSSSVLQWAKKGYY
TMSNTLVTLENGKQLKVKRQGFYYIYAQVTFCSNQEPSSQAPFIASLCLK
SSGGSERILLRAANARSSSKTCEQQSIHLGGVFELQADASVFVNVTDASQ
VNHGTGFTSFGLLKL
SEQ ID NO: 8 C4b multimerization domain; UniProt: P04003; amino acids 540-597
WETPEGCEQVLTGKRLMQCLPNPEDVKMALEVYKLSLEIEQLELQRDSAR
QSTLDKEL
SEQ ID NO: 9-GCN4pII heptad repeat modified from the wildtype dimeric GCN4
repeat found in Saccharomyces cerevisiae
GDRMKQIEDKIEEILSKIYHIENEIARIKKLIGER
SEQ ID NO: 10-wildtype dimeric GCN4 Leucine-
Zipper (P03069, amino acids 253-274)
LEDKVEELLSKNYHLENEVARL
SEQ ID NO: 11-human IgG1 Fc fragment (P01857, amino acids 104-329)
DKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYK
CKVSNKALPAP IEKTISKAKGQPREPQVYTLPPSRDELTKN QVSLTCLVKG
FYPSDIAVEWESNGQPENNYKTTPPVLDSDGSFFLYSKLTVDKSRWQQGN
VFSCSVMHEALHNHYTQKSLSLSPGGGGSGGGGSGPRPPGPX₁X₂
wherein X₁ is no amino acid or G
wherein X₂ is no amino acid or K (in a specific embodiment X₁ and X₂ are no amino
acid, i.e. the sequence ends at position 242)
SEQ ID NO: 12-Fc-CD40-L 18 kDa
DKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNFS
CSVMHEALHNHYTQKSLSLSPGGGGSGGGGSGPRPPGPEINFEMQKGDQD
PQIAAHLISEASSKSSSVLQWAKKGYYTMSNTLVTLENGKQLKVKRQGFY
YIYAQVTFCSNQEPSSQAPFIASLCLKSSGGSERILLRAANARSSSKTCEQQS
IHLGGVFELQADASVFVNVTDASQVNHGTGFTSFGLLKL
SEQ ID NO: 13-LZ-CD40-L EctoD
GDRMKQIEDKIEEILSKIYHIENEIARIKKLIGERTSGGSGGTGGSGGTGGSH
RRLDKIEDERNLHEDFVFMKTIQRCNKGEGSLSLLNCKEIRSQFEGFVKDIM
LNKEEPKKEINFEMQKGDQDPQIAAHLISEASSKSSSVLQWAKKGYYTMS
NTLVTLENGKQLKVKRQGFYYIYAQVTFCSNQEPSSQAPFIASLCLKSSGGS
ERILLRAANARSSSKTCEQQSIHLGGVFELQADASVFVNVTDASQVNHGTG
FTSFGLLKL
SEQ ID NO: 14-LZ-CD40-L EctoD with N-terminal His-tag
HHHHHHHHGDRMKQIEDKIEEILSKIYHIENEIARIKKLIGERTSGGSGGTG
GSGGTGGSHRRLDKIEDERNLHEDFVFMKTIQRCNKGEGSLSLLNCKEIRS
QFEGFVKDIMLNKEEPKKEINFEMQKGDQDPQIAAHLISEASSKSSSVLQW
AKKGYYTMSNTLVTLENGKQLKVKRQGFYYIYAQVTFCSNQEPSSQAPFI
ASLCLKSSGGSERILLRAANARSSSKTCEQQSIHLGGVFELQADASVFVNVT
DASQVNHGTGFTSFGLLKL
SEQ ID NO: 15-LZ-CD40-L 18 kDa domain
LEDKVEELLSKNYHLENEVARLGGGSGGGSGGGEINFEMQKGDQDPQIAA
HLISEASSKSSSVLQWAKKGYYTMSNTLVTLENGKQLKVKRQGFYYIYAQ
VTFCSNQEPSSQAPFIASLCLKSSGGSERILLRAANARSSSKTCEQQSIHLGG
VFELQADASVFVNVTDASQVNHGTGFTSFGLLKL
SEQ ID NO: 16-LZ-CD40-L 18 kDa domain with N-terminal His-tag
HHHHHHHHLEDKVEELLSKNYHLENEVARLGGGSGGGSGGGEINFEMQK
GDQDPQIAAHLISEASSKSSSVLQWAKKGYYTMSNTLVTLENGKQLKVKR
QGFYYIYAQVTFCSNQEPSSQAPFIASLCLKSSGGSERILLRAANARSSSKTC
EQQSIHLGGVFELQADASVFVNVTDASQVNHGTGFTSFGLLKL
SEQ ID NO: 17-C4b-CD40-L
WETPEGCEQVLTGKRLMQCLPNPEDVKMALEVYKLSLEIEQLELQRDSAR
QSTLDKELGGGSGGGSGGGEINFEMQKGDQDPQIAAHLISEASSKSSSVLQ
WAKKGYYTMSNTLVTLENGKQLKVKRQGFYYIYAQVTFCSNQEPSSQAPF
IASLCLKSSGGSERILLRAANARSSSKTCEQQSIHLGGVFELQADASVFVNV
TDASQVNHGT GFTSFGLLKL
SEQ ID NO: 18-C4b-CD40-L with N-terminal His-tag
HHHHHHHHWETPEGCEQVLTGKRLMQCLPNPEDVKMALEVYKLSLEIEQ
LELQRDSARQSTLDKELGGGSGGGSGGGEINFEMQKGDQDPQIAAHLISEA
SSKSSSVLQWAKKGYYTMSNTLVTLENGKQLKVKRQGFYYIYAQVTFCSN
QEPSSQAPFIASLCLKSSGGSERILLRAANARSSSKTCEQQSIHLGGVFELQA
DASVFVNV TDASQVNHGT GFTSFGLLKL
SEQ ID NO: 19-Flexible amino acid linker 1
TSGGSGGTGGSGGTGGS
SEQ ID NO: 20-IL-2 signal peptide
MRRMQLLLLIALSLALVTNS
SEQ ID NO: 21-Ig light chain signal peptide
MGWSCIILFLVATATGVHS
SEQ ID NO: 22-Flexible amino acid linker 2
GGGSGGGSGGG
SEQ ID NO: 23-EPO signal peptide
MGVHECPAWLWLLLSLLSLPLGLPVLGA

### DESCRIPTION OF THE FIGURES

The following figures are provided to aid the understanding of the present invention, the true scope of which is set forth in the appended claims. It is understood that modifications can be made in the procedures set forth without departing from the spirit of the invention.

FIG. 1: SEC-MALS chromatogram received from the miniDAWN TREOS II, Optilab T.rEX Detectors showed a molecular weight of LZ-CD40-L of 89 kDa (=ectodomain of CD40-L). This correlates with a trimeric structure of the LZ-CD40-L EctoD (SEQ ID NO: 14). SEC: Size Exclusion Chromotography, MALS: Multi angle static light scattering.

FIG. 2: SEC-MALS chromatogram received from the miniDAWN TREOS II, Optilab T.rEX Detectors showed a molecular weight of Fc-CD40-L of 300 kDa. This correlates with a hexameric structure of the Fc-CD40-L (SEQ ID NO: 12). SEC: Size Exclusion Chromotography, MALS: Multi angle static light scattering

FIG. 3: SEC-MALS chromatogram received from the miniDAWN TREOS II, Optilab T.rEX Detectors showed a molecular weight of LZ-CD40-L 18 kDa domain (SEQ ID NO: 16) of 63 kDa. This correlates with a trimeric structure of the LZ-CD40-L (18 kDa). SEC: Size Exclusion Chromotography, MALS: Multi angle static light scattering.

FIG. 4: Antibody secreting cell batch sort average growth after 7 days as assessed by cell counts in the media compositions #1-7 in BM1. For the media conditions #1-4 we performed five experiments and for #5-7 four experiments. BM1: Base media 1; TSN: thymocyte supernatant; Fc: human IgG1 Fc (aa104-329); BAFF: soluble B Cell Activating Factor; Cytokines: IL-2 (773 IU/ml), IL-15 (15 ng/ml), IL-21 (20 ng/ml), BAFF (150 ng/ml) and hFc-CD40-L (7.5 pg/ml); “-” indicates which cytokine(s) is/are missing.

FIG. 5: Correlation of B cell batch sort average growth after 7 days with the media compositions #1-7 and the average level of IgG production in BM1. (N=2) BM1: Base media 1; TSN: thymocyte supernatant; Fc: human IgG1 Fc (aa104-329); BAFF: soluble B Cell Activating Factor; Cytokines: IL-2 (773 IU/ml), IL-15 (15 ng/ml), IL-21 (20 ng/ml), BAFF (150 ng/ml), hFc-CD40-L (7.5 μg/ml); “-” indicates which cytokine(s) is/are missing.

FIG. 6: B cell batch sort average growth after 7 days using the media compositions #8-12 in BM2. (N=3) BM2: Base media 2; TSN: thymocyte supernatant; Fc: human IgG1 Fc (aa104-329); BAFF: soluble B Cell Activating Factor; Cytokines: IL-2 (773 IU/ml), IL-15 (15 ng/ml), IL-21 (20 ng/ml), BAFF (150 ng/ml) and hFc-CD40-L (7.5-2.5 μg/ml).

FIG. 7: Comparison of the use of CD40-L EctoD and CD40-L 18 kDa Fragments in BM1 (N=3) after 7 days of culture BM1: Base Media 1; TSN: thymocyte supernatant; Fc: human IgG1 Fc (aa104-329); LZ: leucine zipper (note LZ used are different in amino acid sequences); BAFF: soluble B Cell Activating Factor; Cytokines: IL-2 (773 IU/ml), IL-15 (15 ng/ml), IL-21 (20 ng/ml), BAFF (150 ng/ml); EctoD: Ectodomain (aa47-261); 18 kDa: (aa108-261); CD40-L constructs with 7.5 μg/ml.

FIG. 8: Average antigen specific ELISA hits in single cell sort experiments in BM1 and BM2 after 7 days of culture. (N=8) BM1: Base Media 1; BM2: Base Media 2; TSN: thymocyte supernatant; Fc: human IgG1 Fc (aa104-329); BAFF: B Cell Activating Factor; Cytokines: IL-2 (773 IU/ml), IL-15 (15 ng/ml), IL-21 (20 ng/ml), and soluble BAFF (150 ng/ml); 18 kDa: (aa108-261); hFc-CD40-L (7.5 μg/ml).

FIG. 9: Representative gating strategy for isolating plasmablasts. This flow cytometry blot is representative for the gating strategy used to sort IgGi^{int}FSCi^{int} B cells (i.e. plasmablasts) from PBMCs that have been previously selected for antigen binding via MACS. Cells depicted have been gated for living lymphocytes via 7-AAD and FSC/SSC. Memory B cells show high levels of cell surface IgG and are small in size, whereas plasmablasts show intermediate levels of cell surface IgG and are intermediate in size.

### EXAMPLES

The following examples are provided to aid the understanding of the present invention, the true scope of which is set forth in the appended claims. It is understood that modifications can be made in the procedures set forth without departing from the spirit of the invention.

### Example 1: Immunization of Rabbits with Antigens and Isolation and Deposition of Primary Antibody Expressing Cells from Peripheral Blood Obtained of Said Rabbits

### Immunization of Animals

In the following Examples isolated antibody secreting cells obtained from peripheral blood of rabbits immunized with different immunogens were employed (in total 8 to 11 different immunogens each for deriving antibodies against completely unrelated target antigens). The data presented in the following examples is based on average data obtained from independent experiments, i.e. independently analyzed rabbit blood samples, including also blood samples from immunizations with different immunogens.

For each immunization, 12-16 weeks old NZW (New Zealand White) rabbits were immunized with the respective immunogen. Animals were immunized weekly in the first month and subsequently once per month. The first immunization was performed “with Complete Freund's Adjuvant (CFA) and the following immunizations with Incomplete Freund's Adjuvant (IFA).

Isolation of Antibody Expressing Cells from Peripheral Blood Obtained from Antigen-Immunized Rabbits and Staining for Subsequent FACS Sorting and Deposition

Peripheral blood from each rabbit immunized with an immunogen of interest was obtained for isolating antigen-specific antibody expressing cells.

For the antibody expressing cell isolation all working reagents were cooled to a temperature of 4° C. Moreover, all incubation steps were conducted at 4° C. Centrifugation was performed at 4° C. and 600 g for 10 minutes (min) with terminal break, if not noted otherwise.

First, PBMCs (i.e. lymphocytes and monocytes) were isolated from rabbit EDTA blood. Rabbit EDTA blood was diluted 1:1 with PBS (Gibco, 10010-015) and distributed to Leucosep™ Tubes (Greiner bio-one, 227288) at a volume of 20-30 ml. The centrifugation was performed at 800 g for 15 min without terminal break. After transferring the interphases of one animal into one collection tube (Falcon, 352075) with 130 ml of PBS, the cells were centrifuged for 15 min. For precise cell count, the cell pellet was resuspended in 40 ml PBS. Cell count was conducted using C-Chip™ Fuchs-Rosenthal Counting Chambers (NanoEnTek, DHC-FO1), after diluting a sample of 90 μl 1:10 with Trypan Blue (Gibco, 15250061; working concentration: 0.08%). The remaining cells were centrifuged in a SuperClear tube (VWR, 525-0156).

In a second step, antigen-specific antibody expressing cells were enriched from the pelleted PBMCs. To prepare the PBMCs for enrichment of antibody expressing cells, the PBMC pellet was resuspended in 1 ml FACS (Table 1) buffer comprising biotin-conjugated antigen (the antigen corresponding to the respective immunogen used for immunization of the animal) per 5.0×10⁷ cells and the mixture was incubated for 25 min to capture the antibody expressing cells. To wash the cells, the solution was filled up to 40 ml with PBS and centrifuged. The pellet was resuspended in 90 μl labeling buffer and 10 μl Streptavidin MicroBeads (Miltenyi Biotec, 130-048-101) per 1.0×10⁷ cells. During 15 min of incubation, the magnetic beads are able to bind to the biotin-conjugated antigen and the antibody expressing cells bound thereto. The mixture was washed by filling up the solution to 40 ml with PBS and centrifuging. For the enrichment step, the pellet was resuspended in 500 μl MACS buffer per 1.0×10⁸ cells. To separate the antibody expressing cells from the rest of the cells, the sample was passed through a magnetized LS column (Miltenyi Biotec, 130-042-401) and rinsed with 3 ml MACS buffer for three times. By washing the column with 5 ml MACS buffer without a magnetic field, the antibody expressing cells were eluted into a SuperClear tube (VWR, 525-0150).

### Staining, Sorting and Deposition of Antibody Expressing Cells (in Particular Plasmablasts)

The antigen-specific antibody expressing cells obtained with the protocol detailed above were next stained with an anti-Rabbit-IgG antibody and 7-AAD to be able to select surface IgG⁺ and viable cells via FACS sorting.

To this end, the antibody expressing cells were pelleted by centrifugation, transferred into a round-bottom tube (Falcon, 352054), and dyed for 30 min with 500 μl FACS buffer, containing FITC-labeled monoclonal mouse anti-Rabbit-IgG antibody (Southern Biotech, 4090-02) at a concentration of 1 μg/ml at 4° C. In a final centrifugation step for 5 min, the cells were washed with 2 ml PBS and resuspended in 500 μl FACS buffer. 5 min before sorting 1 μl 7-AAD per 100 μl FACS buffer was added.

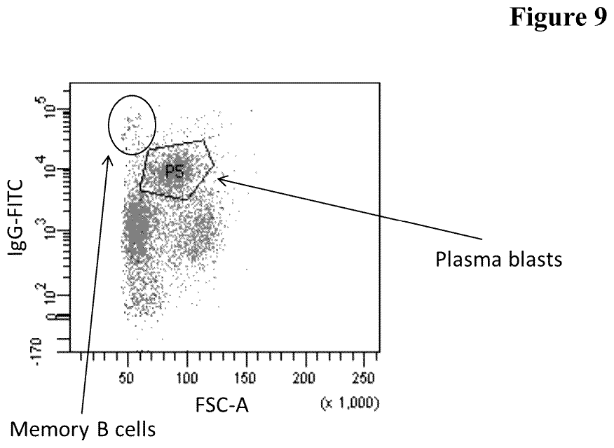

The stained cells were next sorted and deposited with a Sony Biotechnology SH-800S cell sorter. Specifically, plasmablasts were selected by gating for IgGi^{int}FSC^{int} cells. A representative gating strategy for the gating strategy to deposit plasmablasts is shown in FIG. 9.

For “batch sort experiments” (i.e. the experiments in which 50 or 100 cells were deposited per well), a seeding culture was prepared. For this purpose 2600 IgG positive and 7-AAD negative cells were deposited into 2.6 ml base media (37° C.). 50 μl of the seeding culture was transferred to each concentrated media condition (1.36×) with 140 μl. In this way a start plasmablast number of 50 cells in 190 μl per well with the final media concentrations was achieved.

For “single cell” experiments, the plasmablasts were cultured in 190 μl per well (if feeder cells were used) or in 70 μl per well (for the recombinant settings without feeder cells). The entire volume was submitted into the plates and the plasmablasts were deposited with a Sony Biotechnology SH-800S cell sorter directly into the wells.

TABLE 1
Buffers used in Example 1

| | | | Catalog | |
|---|---|---|---|---|
| Buffer | Compound | Supplier | Number | Concentration |
| FACS | PBS | Oxoid | BR0014G | 1x |
| | BSA | Roche | 3535240001 | 0.1% |
| Panning | FACS Buffer | — | — | 1x |
| | Antigen | — | — | 150-250 ng/ml |
| Labeling | PBS | Oxoid | BR0014G | 1x |
| | EDTA | Merck | 1.08418 | 2 mM |
| MACS | PBS | Oxoid | BR0014G | 1x |
| | BSA | Roche | 3535240001 | 0.5% |
| | EDTA | Merck | 1.08418 | 2 mM |

### Example 2: Expression and Purification of the CD40 Ligand Constructs

For some experiments in the Examples below different recombinantly expressed CD40 Ligand (CD40-L) constructs were expressed:

The first molecule, LZ-CD40-L (SEQ ID NO: 14, also referred to as LZ-CD40-L Ectodomain or LZ-CD40-L EctoD, features an octahistidine tag, a trimeric leucine zipper GCN4pII heptad repeat (LZ) (Naito, M. H. (2013). Cancer Immunol Immunother(62), pp. 347-357.) obtained by modification of the wildtype dimeric GCN4 repeat found in Saccharomyces cerevisiae (GDRMKQIEDKIEEILSKIYHIENEIARIKKLIGER; SEQ ID NO: 9), a flexible 17 amino acid linker (TSGGSGGTGGSGGTGGS; SEQ ID NO: 19), and the entire extracellular domain of rabbit CD40-L (G1SKP7, amino acids 47-261; SEQ ID NO: 7). The LZ-CD40-L construct was expressed by transient transfection using the FreeStyle 293 Expression System according to the manufacturers instructions (Thermo Fisher Scientific, Waltham, MA, USA). For the expression, an N-terminal IL-2 signal peptide (MRRMQLLLLIALSLALVTNS; SEQ ID NO: 20) was used, which is cleaved of during secretion.

The LZ-CD40-L was purified via the His-Tag with a Ni-NTA column (Superflow, Qiagen) and concentrated with an Amicon® 10 kDa (Merck) centrifugal device. The last purification step was conducted with a preparative Superdex™ 200 column (GE Healthcare).

The analytics was performed with SEC (Size Exclusion Chromatography) (Thermo Fischer, Ultimate 3000 UHPLC) and a MALS (Multi angle static light scattering) Detector (miniDAWN TREOS II, Optilab T.rEX; Wyatt Technologies).

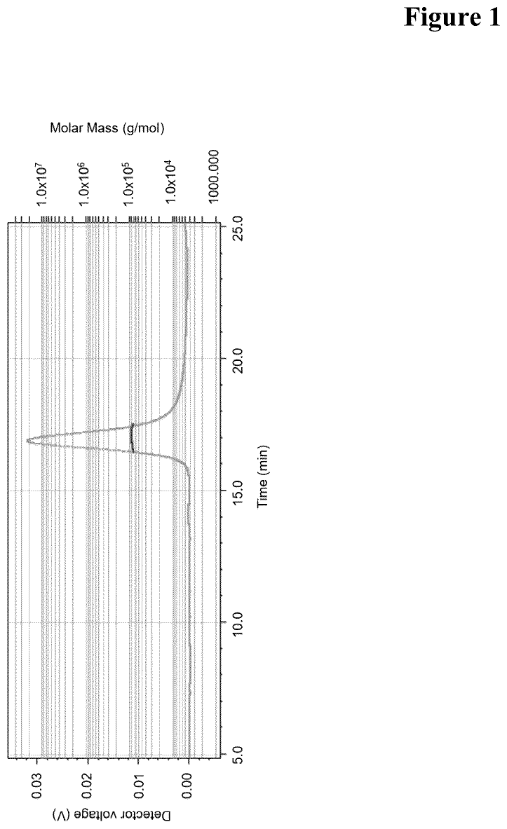

After purification, the LZ-CD40-L showed a trimeric structure with a molecular weight of 89 kDa (FIG. 1, mark in the peak).

The second molecule Fc-CD40-L(SEQ ID NO: 12), also referred to as FC-CD40-L 18 kDa featured a human IgG1-Fc fragment (SEQ ID NO: 11 with X₂ being no amino acid) linked to a 18 kDa rabbit CD40-L fragment (GISKP7, aa108-261; SEQ ID NO: 1). For the expression, an N-terminally fused Ig light chain signal peptide (MGWSCIILFLVATATGVHS; SEQ ID NO: 21) was employed, which is cleaved of prior to secretion. The Fc-CD40-L construct was expressed by transient transfection using the FreeStyle 293 Expression System according to the manufacturers instructions (Thermo Fisher Scientific, Waltham, MA, USA). The 18 kDa CD40-L was defined by sequence comparison with human CD40-L and the corresponding definition of an 18 kDa CD40-L fragment found to have CD40-L activity (Durandy et al., Journal of Biological Chemistry, Volume 270, Issue 13, Pages 7025-7028, 1995).

The Fc-CD40-L 18 kDa was purified with a MabSelect™ SuRe™ (GE Healthcare) via the human IgG1 Fc-Tag and concentrated with an Amicon® 10 kDa (Merck) centrifugal device. A second purification step was conducted with a preparative Superdex™ 200 column (GE Healthcare). The analytics was performed with SEC (Size Exclusion Chromatography) (Thermo Fischer, Ultimate 3000 UHPLC) and a MALS (Multi angle static light scattering) Detector (miniDAWN TREOS II, Optilab T.rEX; Wyatt Technologies).

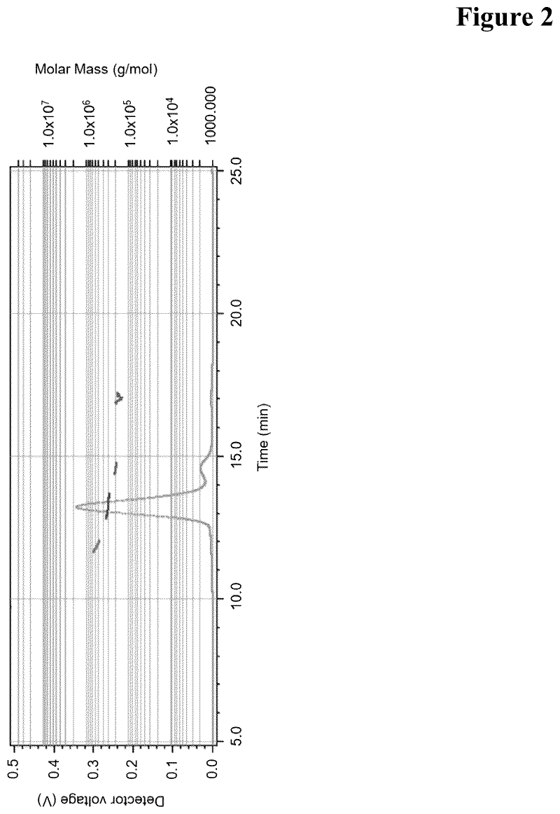

After purification, the Fc-CD40-L showed a hexameric structure with a molecular weight of 300 kDa (FIG. 2, mark in the first peak)

The third molecule, LZ-CD40-L(18 kDa), SEQ ID NO: 16, featured) an octahistidine tag, a GCN4 Leucine-Zipper (LZ) motif (P03069, amino acids 253-274), a flexible GGGSGGGSGGG (SEQ ID NO: 22) amino acid linker fused to the 18 kDa rabbit CD40-L fragment (G1SKP7, amino acids 108-261; SEQ ID NO: 1). The construct was expressed with an N-terminal an EPO signal peptide (MGVHECPAWLWLLLSLLSLPLGLPVLGA; SEQ ID NO: 23) which was cleaved of during secretion. The LZ-CD40-L(18 kDa) was expressed by transient transfection using the FreeStyle 293 Expression System according to the manufacturers instructions (Thermo Fisher Scientific, Waltham, MA, USA).

The LZ-CD40-L (18 kDa) was purified via the His-Tag with a Ni-NTA column (Superflow, Qiagen) and concentrated with an Amicon® 10 kDa (Merck) centrifugal device. The last purification step was conducted with a preparative Superdex™ 200 column (GE Healthcare).

The analytics was performed with SEC (Size Exclusion Chromatography) (Thermo Fischer, Ultimate 3000 UHPLC) and a MALS (Multi angle static light scattering) Detector (miniDAWN TREOS II, Optilab T.rEX; Wyatt Technologies).

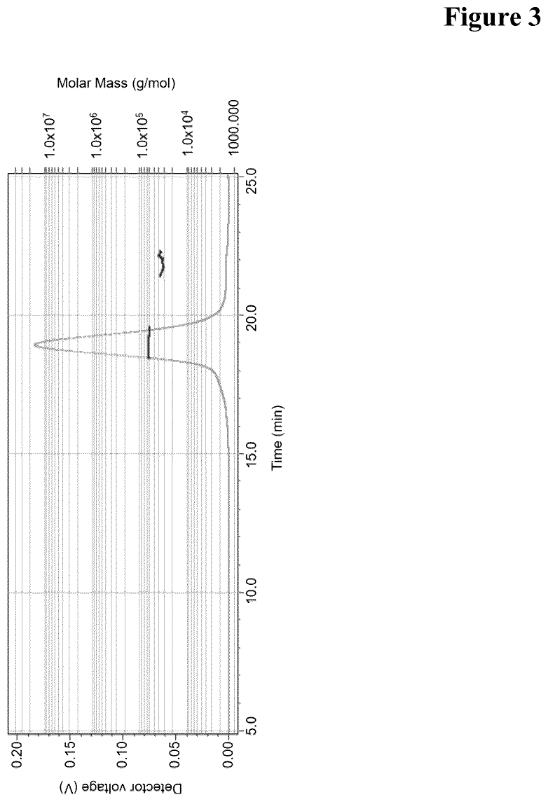

After purification, the LZ-CD40-L (18 kDa) showed a trimeric structure with a molecular weight of 63 kDa (FIG. 3, mark in the peak).

The final working concentration of LZ-CD40-L and LZ-CD40-L (18 kDa) in the cell culture medium was 7.5 μg/ml and the Fc-CD40-L was used at final working concentrations from 2.5 to 7.5 μg/ml in the cell culture medium.

### Example 3: Identification of Recombinant Factors to Replace TSN and Feeder Cells in the Culture System for Antibody Expressing Cells

In state of the art, single cell culturing and proliferation systems for primary antibody expressing cells, such as plasmablasts, obtained from peripheral blood of rabbits have typically employed a feeder cell based system (using e.g. EL4-B5 cells) and a conditioned cell culture supernatant (typically thymocyte supernatant (TSN)) to achieve a high proliferation efficiency of primary antibody expressing cells (Seeber et al, PLoS One. 2014 Feb. 4; 9(2):e86184. doi: 10.1371/journal.pone.0086184).

As discussed herein above, using conditioned cell culture supernatant and feeder cells for proliferating antibody expressing cells comes with certain limitations.

The aim of this study was to explore the possibility of fully recombinant culture settings for antibody expressing cells to address these limitations, which is at least comparable efficient in proliferation efficiency of antibody expressing cells to the TSN and feeder cell base culturing system.

We hypothesized that certain secreted cytokines in the TSN as well as ligands or receptors provided to the antibody expressing cells in cell-cell contacts with the feeder cells are the key factors for the proliferation of antibody expressing cells in ex vivo culture. Thus, the capability of numerous different culturing conditions using different cytokine combinations and different feeder cell replacements were analyzed in a “batch sort” pre-screening, in which the proliferation of cell pools of 100 (first DoE) or 50 cells (second DoE) per well was evaluated after 7 days of culture. For the pre-screening, different cytokines, ligands and receptors were evaluated in two DoE (Design of Experiments) based experiments: (i) a first DoE for assessing combinations of soluble factors for replacing TSN without replacing feeder cells; and (ii) a second DoE for assessing different combinations of factors to replace TSN and feeder cells.

The “batch sort” experiments employed for the DoE were conducted with 100 plasmablasts from peripheral blood per well (isolated according to Example 1).

The cultivation duration was seven days at 37° C. and 5.0% CO₂.

For the DoE batch sort growth cultures two different B cell media were used. The first media used for the first DoE experiment (referred to as BM1) was based on RPMI 1640 (Gibco, 31870) media that contained 5% fetal calf serum (FCS; Gibco 16149), 292 μg/ml Glutamine (Gibco, 10378), 2 mM Pyruvate (Gibco, 11360), 24 pM B-Mercoptoethanol (Gibco, 31350) and 10 mM HEPES (Gibco, 15630). The second media used for the second DoE (BM2) was based on eRDF media (Murakami H, 1989, Advances in Biotechnological Processes, Vol. 11, Monoclonal Antibodies: Production and Application. Alan R. Liss, New York, pp. 107-141). It contained 5% fetal calf serum (Gibco 16149), 877 μg/ml Glutamine (Sigma Aldrich, G5792) and 24 pM B-Mercoptoethanol (Gibco, 31350). Before the antibody expressing cell cultivation started, SAC (Staphylococcus aureus cells; Merck, 507858; 1:19.500) was added to the BM1 and BM2.

The read out of the batch sort DoE experiments was the determination of the antibody expressing cell, in particular plasmablasts, number in the respective well. For quantification of antibody expressing cell growth in batch sort, a Sony Biotechnology SH-800S cell sorter was used and the cell number of each well after seven days of culture was determined. The parameters were BSC (Side Scatter also referred to as SSC) vs. FSC (Forward Scatter) for the separation of the EL4-B5 cells from the antibody expressing cells. Before the quantification step was carried out 50 μl supernatant (without cells) of each culture was transferred to a new 96 well plate for the IgG quantification (N=4). The remaining 140 μl of culture were used undiluted for the cell number quantification.

The first DoE for the TSN replacement was done with hIL-2 (0-1.000 IU/ml, Roche), hIL-4 (0-50 ng/ml, PeproTech, 200-04), hIL-5 (0-20 ng/ml, PeproTech, 200-05), hIL-6 (0-50 ng/ml, produced in house), hIL-10 (0-50 ng/ml, PeproTech, 200-10), hIL-12 (0-10 ng/ml, PeproTech, 200-12H), hIL-15 (0-15 ng/ml, PeproTech, 200-15), hIL-21 (0-20 ng/ml, PeproTech, 200-21), hBAFF (0-150 ng/ml, R&D Systems, 7537-BF-025), hAPRIL (A proliferation-inducing ligand (0-150 ng/ml, R&D Systems, 5860-AP-010)) and hTNFu (tumor necrosis factor alpha (0-5 ng/ml, PeproTech, 300-01A)) in 28 different combinations and 6 experiment replicates.

Table 2 shows all terms which were significant (“Prob>|t|”<0.05) for the model of this first DoE. All terms without significance (“Prob>|t|”>0.05) were excluded from the model. The strongest positive impact on proliferation of the antibody secreting cells had IL-2, IL-15, IL-21 and BAFF. APRIL had a small positive impact, but a low t Ratio with 2.70. IL-4 showed a negative impact on the cell proliferation.

TABLE 2
Parameter Estimates with significance
of DoE for the TSN replacement

| Term | Estimate | Std. Error | t Ratio | Prob > \|t\| |
|---|---|---|---|---|
| IL-2 | 0.0109126 | 0.001106 | 9.86 | <.0001 |
| IL-4 | −0.068132 | 0.020277 | −3.36 | 0.0009 |
| IL-15 | 0.2666173 | 0.062804 | 4.25 | <.0001 |
| IL-21 | 0.1563906 | 0.045235 | 3.46 | 0.0006 |
| BAFF | 0.0317688 | 0.007168 | 4.43 | <.0001 |
| APRIL | 0.016261 | 0.00603 | 2.70 | 0.0074 |

(DoE: Design of Experiments; TSN: Thymocyte Supernatant; BAFF: B Cell Activating Factor; APRIL: A proliferation inducing ligand)

The second DoE for the feeder cell and TSN replacement was performed with hIL-2 (0-10.000 IU/ml produced in-house), hIL-4 (0-50 ng/ml, PeproTech, 200-04), hIL-6 (0-100 ng/ml, produced in-house), hIL-10 (0-100 ng/ml, PeproTech, 200-10), hIL-13 (0-50 ng/ml, PeproTech, AF-200-13), hIL-21 (0-50 ng/ml, PeproTech, 200-21), hBAFF soluble form (0-150 ng/ml, R&D Systems, 7537-BF-025), hAPRIL (0-150 ng/ml, R&D Systems, 5860-AP-010), hTNFu (0-100 ng/ml, PeproTech, 300-01A), hOX40 (0-10.000 ng/ml, R&D Systems, 3388-OX-050), hICOS (inducible co-stimulator (0-10.000 ng/ml, R&D Systems, 169-CS-050)), hCD28 (0-10.000 ng/ml), hSema4D (0-10.000 ng/ml, R&D Systems, 7470-S4-050), hCD27 (0-10.000 ng/ml, R&D Systems, 382-CD-100), h4-1BB (0-10.000 ng/ml, R&D Systems, 838-4B-100), h4-1 BBL (0-10.000 ng/ml, PeproTech, 310-11), rabbit CD40-L (0-10.000 ng/ml, Roche, LZ-CD40-L) and PMA (Phorbol-12-myristat-13-acetat (0-5 ng/ml, Sigma Aldrich, 880134P)) in 32 different combinations and 6 experiment replicates. In this DoE the cytokines were combined with cell surface proteins to see, whether these proteins have an impact on the cell growth and the cytokine combination as well as the confirmation of the cytokines from the first DoE (i). In this DoE 50 plasmablasts were used per cultivation well.

As in the first DoE, all terms in the second DoE without a significant (“Prob>|t|”>0.05) impact were excluded (Table 3). Compared to the first DoE APRIL had no significant impact on the cell proliferation. Moreover, ICOS, 4-1BB and PMA showed a negative impact, which is significant. Only CD40-L showed a positive and significant impact of the terms for feeder cell replacement. Furthermore, the second DoE confirmed the positive significant impact of IL-2, IL-21 and BAFF on the cell proliferation.

TABLE 3
Parameter Estimates with significance
of DoE for the EL4-B5 replacement

| Term | Estimate | Std. Error | t Ratio | Prob > \|t\| |
|---|---|---|---|---|
| IL-2 | 149.94496 | 30.31978 | 4.95 | <.0001 |
| IL-21 | 56.986219 | 24.32387 | 2.34 | 0.0296 |
| BAFF | 62.09129 | 24.79631 | 2.50 | 0.0211 |
| ICOS | −54.33103 | 24.68151 | −2.20 | 0.0396 |
| 4-1BB | −79.2649 | 24.63577 | −3.22 | 0.0043 |
| Rabbit CD40-L | 139.26163 | 31.22131 | 4.46 | 0.0002 |
| PMA | −61.09388 | 25.70751 | −2.38 | 0.0276 |

(DoE: Design of Experiments; BAFF: B Cell Activating Factor; ICOS: Inducible co-stimulator APRIL: A proliferation inducing ligand; PMA: Phorbol-12-myristat-13-acetat)

The results of the two DoEs were combined and revealed IL-2, IL-15, IL-21 and BAFF as promising factors for TSN replacements and soluble CD40 ligand as a feeder cell replacement candidate.

### Example 4: Further Evaluation of the Recombinant Factors to Replace TSN and Feeder Cells in the Batch Sort Culture System for Antibody Expressing Cells Using BM1 Medium

The candidates for TSN replacement (IL-2, IL-15, IL-21 and BAFF) and feeder cell replacement (CD40-L) identified in the DoE pre-screening of Example 3 were studied in more detail and with an increased amount of samples in “batch sort experiments”, in which the proliferation of cell pools of 50 cells per well was evaluated. In brief, antibody expressing cells were isolated from freshly isolated PBMCs by antigen panning and IgG positive (IgG^{int}, SSC^{int} and 7-AAD negative cells were selected and deposited as batch (50 cells per well) into culture medium as described in Example 3, above. Subsequently, the cells were cultured in batch, as described in Example 3 above, in presence of different cytokine combinations and in the presence of feeder cells or soluble CD40-L constructs in BMT medium.

The following factor combinations were used:

TABLE 4
Media combinations for the B cell batch sort
growth experiments with Base Media 1 (BM1);

| | Setting/ | | | | | | | |
|---|---|---|---|---|---|---|---|---|
| # | Compound | EL4-B5 | TSN | IL-2 | IL-15 | IL-21 | BAFF | Fc-CD40-L |
| 1 | Blank | — | — | — | — | — | — | — |
| 2 | TSN + | X | X | — | — | — | — | — |
| | EL4-B5 | | | | | | | |
| 3 | Cytokines + | X | — | X | X | X | X | — |
| | EL4-B5 | | | | | | | |
| 4 | Cytokines + | — | — | X | X | X | X | X |
| | Fc-CD40-L | | | | | | | |
| | (7.5 μg/ml) | | | | | | | |
| 5 | Cytokines + | — | — | X | X | X | — | X |
| | Fc-CD40-L | | | | | | | |
| | (7.5 μg/ml)- | | | | | | | |
| | BAFF | | | | | | | |
| 6 | Cytokines + | — | — | X | — | X | — | X |
| | Fc-CD40-L | | | | | | | |
| | (7.5 μg/ml)- | | | | | | | |
| | BAFF-IL-15 | | | | | | | |
| 7 | Cytokines + | — | — | X | X | — | — | X |
| | Fc-CD40-L | | | | | | | |
| | (7.5 μg/ml)- | | | | | | | |
| | BAFF-IL-21 | | | | | | | |

X indicates presence;
— indicates absence

To quantify cell growth in BM1, a seeding culture with 2.600 antibody expressing cells in 2.6 ml was prepared for each experiment. 50 μl of the seeding culture was transferred to each media composition (Table 4) and was performed in three replicates per experiment. For the media conditions #1-4 were performed five experiments and for #5-7 four experiments.

The read out was determination of cell numbers after 7 days of culturing (see Example 3 above).

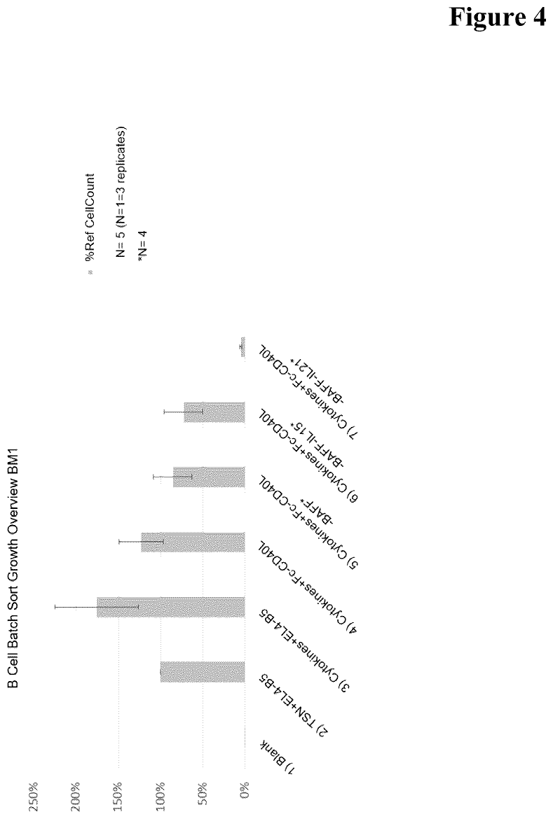

FIG. 4 shows that there was no cell growth in the blank conditions (i.e. BM1 medium only). The value of the reference condition with TSN+EL4-B5 (#2) was set as 100%. In comparison to the reference cell growth in #3 with EL4-B5+ cytokines was approximately 75% higher, showing that TSN can be efficiently replaced.

In relation to the experiments with cytokines+EL4-B5 (#3) the growth of #4 was 1.4 times lower. The cell growth in the media conditions #4 was 23% higher as with the reference conditions (#1).If BAFF was missing in the cytokine composition (#5), cell proliferation was ~1.4 times lower than with all four cytokines (#4) and 85% of the reference (#2). The growth reduction without BAFF and IL-15 (#6) vis-i-vis condition #5 was low. The cell proliferation in #6 was 27% lower than the reference and −1.7 times lower than with all cytokines (#4). When only IL-2 and IL-15 were used as cytokines (#7), there was a high growth reduction. The cell growth was only 5% of the reference (#2). This data demonstrates that with IL-2 and IL-21 alone already a high proliferation efficiency can be achieved. The efficiency is further improved by BAFF and/or IL-15.

To confirm that the cultured cells still allow for antibody secretion (i.e. that the increase in cell numbers in FIG. 4 corresponds to a proliferation of antibody secreting cells), the IgG level in the supernatant of the cells was assessed.

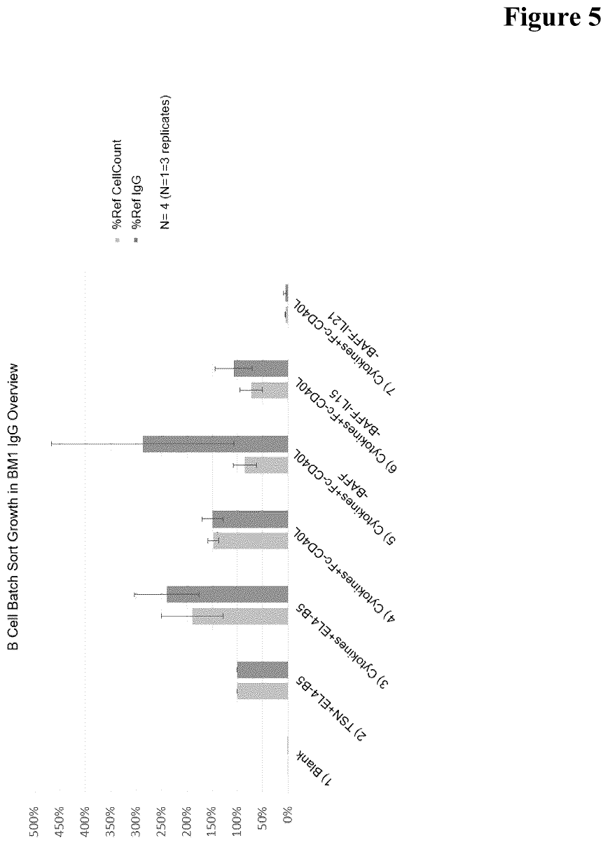

Specifically, in four of the experiments the IgG concentration from each replicate of eight (#1-7) different media compositions (for each composition 12 IgG values) was quantified (FIG. 5).

The rabbit IgG quantification was conducted with an anti-rabbit specific IgG ELISA. The ELISA buffer contained 1.0% BSA (Roche, 3535240) and PBS (Gibco, 10010-015). All steps were performed at room temperature for 1 hour. The wash procedure included three wash steps with 300 μl wash buffer. The wash buffer contained 0.9% NaCl (Merck, 1064045000) and 0.05% Tween20 (Sigma Aldrich, 8.22184). 96-well Streptavidin precoated plates, which were incubated with a biotin conjugated polyclonal goat anti-rabbit IgG with 500 ng/ml (Sigma-Aldrich; B8895), were used. After the wash procedure, the plates were incubated with the cell culture supernatants in different dilutions. The samples were diluted with ELISA buffer. After another washing step the plates were incubated with a polyclonal goat anti-rabbit IgG-Fc at a 1:12.500 dilution. Afterwards, the last wash procedure and the incubation with ABTS (Roche, 10102946001) for 10 minutes followed. The detection wave length was 405 nm and the reference wave length was 492 nm.

The values for cell growth and IgG level of the reference condition with TSN+EL4-B5 (#2) was set as 100%. In all media settings the IgG expression level correlates well with the growth level confirming that the antibody secreting cells proliferated and remained IgG producing cells. This data confirmed that the antibody secreting cells proliferate and showed that the cell numbers are a sufficient measure for assessing the cultivation.

The results described above and shown in FIGS. 4 and 5 confirmed that primary antibody expressing cells and in particular antibody secreting cells such as primary plasmablasts could be efficiently proliferated ex vivo in absence of feeder cells and TSN. The results support that by using a defined cytokine mixture comprising at least IL-2 and IL-21 and a soluble CD40-L comprising an 18 kDa fragment of the extracellular domain (aa108-261) with an Fetag (hexameric structure) can efficiently trigger cell survival and proliferation in the absence of feeder cells and TSN.

### Example 5: Evaluation of the Recombinant Factors to Replace TSN and Feeder Cells in the Batch Sort Culture System in BM2 Medium

To confirm that the conditions for antibody expressing cell proliferation, identified in Example 1, were independent of the basal medium, we used a second set of batch sort experiments, which was conducted in BM2 medium. Three experiments using the BM2 medium and the media compositions as summarized in table 5 were conducted.

TABLE 5
Media combinations for the antibody expressing cell
batch sort growth experiments with Base Media 2 (BM2)

| | Setting/ | | | | | | | |
|---|---|---|---|---|---|---|---|---|
| # | Compound | EL4-B5 | TSN | IL-2 | IL-15 | IL-21 | BAFF | Fc-CD40-L |
| 8 | Blank | — | — | — | — | — | — | — |
| 9 | TSN + | X | X | — | — | — | — | — |
| | EL4-B5 | | | | | | | |
| 10 | Cytokines + | — | — | X | X | X | X | X |
| | Fc-CD40-L | | | | | | | |
| | (7.5 μg/ml) | | | | | | | |
| 11 | Cytokines + | — | — | X | X | X | X | X |
| | Fc-CD40-L | | | | | | | |
| | (5.0 μg/ml) | | | | | | | |
| 12 | Cytokines + | — | — | X | X | X | X | X |
| | Fc-CD40-L | | | | | | | |
| | (2.5 μg/ml) | | | | | | | |

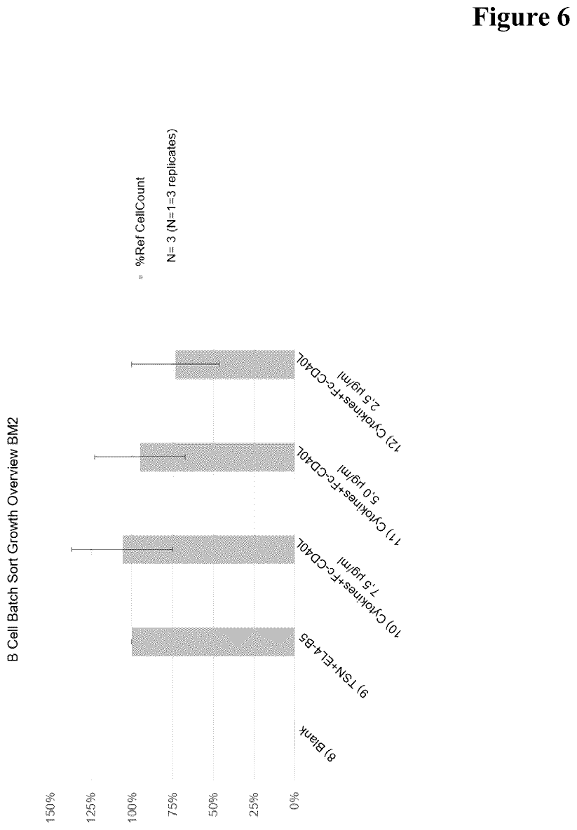

FIG. 6 shows that there was no cell growth in the blank conditions after 7 days, as expected. The value of the reference condition with TSN+EL4-B35 (#9) was set as 100%. Compared to the reference cell growth in #10 with recombinant cytokines (IL-2, IL-15, IL-21 and BAFF)+7.5 μg/ml Fc-CD40-L was, with an approximately 6% higher value, on a comparable level.

This confirms that the culture setting as identified in Examples 3 and 4 can also be used in another B cell culture media in absence of feeder cells and TSN.

Moreover, it was tested which impact the concentration of the Fc-CD40-L had on the B cell growth. With a 50% lower Fc-CD40-L (#11) concentration (5.0 μg/ml) the cell growth was only 5% lower as the reference #9 and ~1.1 times lower than #10 with 7.5 μg/ml. A further reduction of Fc-CD40-L by 50% to 2.5 μg/ml resulted in a 27% lower B cell growth in relation to the reference #9. Compared to #10 with 7.5 μg/ml the cell growth with 2.5 μg/ml was ~1.4 times lower. Despite this small reduction, the results demonstrate that CD40-L can be used over a wide concentration range and that a skilled person can easily adapt the concentration depending on the needs.

### Example 6: Comparison of Different Soluble CD40-L Constructs

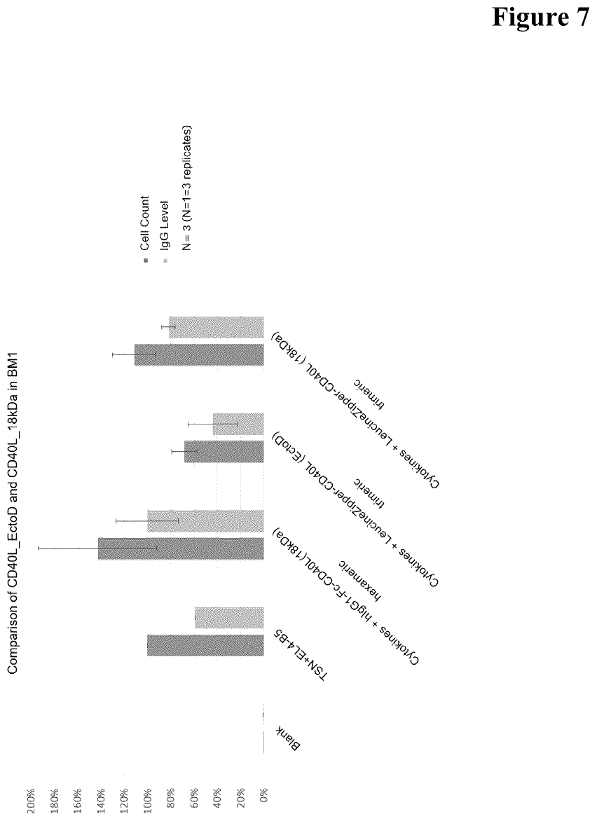

To confirm that the results of the examples above can repeat with different kinds of CD40-L multimerization constructs and with different CD40-L protein fragments, 3 independent experiments were conducted with 3 different CD40-L constructs (see FIG. 7). This was done in “batch sort experiments”, in which the proliferation of cell pools of 50 cells per well was evaluated after 7 days of culturing. In brief, antigen specific plasmablasts were isolated by antigen panning and IgG positive (IgG^{int} and 7-AAD negative cells were selected and deposited as batch (i.e. 50 cells per well) into culture medium as described in Example 3, above. Subsequently, the cells were cultured in batch, as described in Example 3 above, in presence of cytokines (IL-2, IL-15, IL-21 and BAFF) or TSN and in the presence of feeder cells or soluble CD40-L constructs in BM1 medium.

The first construct, consisting of hIgG1-Fc (aa104-329) linked to a rabbit CD40-L fragment (aa108-261) achieved the best growth of all three constructs (see FIG. 7). It exhibited 43% more B cell growth as in the reference (EL4-B5+TSN) and 32% higher B cell growth as the second best construct LZ-CD40-L (18 kDa). The combination of LZ-CD40-L EctoD, consisting of the trimeric leucine zipper linked to the rabbit CD40-L ectodomain, achieved the lowest growth with 68% of the reference.

These results confirms that the evaluated combination of cytokines with different CD40-L fragments and different multimerization domains can replace the standard procedure with EL4-B5 cells and TSN. However, it also demonstrates that the hexameric construct with the hIgG1-Fc as multimerization domain (i.e. leading to a hexameric construct, see above) and the 18 kDa CD40-L fragment was the best among all tested soluble CD40-L constructs.

### Example 7: Antibody Expressing Cell Growth after Single Cell Deposition

To demonstrate that the conditions identified in the batch-sort experiments (see Examples 3 to 5) can be successfully used on a single-cell level, single-cell deposition experiments were performed. In total, eight experiments were conducted and results are shown as average.

Specifically, antibody expressing cells (in particular plasmablasts) were isolated and IgG positive (int) and 7-AAD negative cells were selected (as described in Example 1) and deposited as single cells. Single antibody expressing cells were deposited with a Sony Biotechnology SH-800S cell sorter in 190 μl (EL4-B5+TSN BM1, 96 well plate) medium per well and in 70 μl (cytokines+Fc-CD40-L BM2, 384-well plate) per well for the recombinant setting. The entire volume was submitted into the plates and the single antibody expressing cells were deposited into the wells.

The single cell cultures were conducted in two settings for 7 days. The reference setting (EL4-B5+TSN BM1) with 1.0 ml TSN per 18.5 ml BM1 and 105.263 cells/ml 50 Gy radiated EL4-B5 cells in BM1 medium as well as the recombinant setting (Cytokines+Fc-CD40-L in BM2 medium) with IL-2 (773 IU/ml), IL-15 (15 ng/ml), IL-21 (20 ng/ml), BAFF (150 ng/ml) and Fc-CD40-L (7.5 μg/ml) in BM2. Both settings contained SAC 1:19.500.

The antibody expressing cells in the reference setting were cultured in four 96-well plates (Thermo Fisher Scientific, 161093) and the antibody expressing cells in the recombinant setting in one 384-well plate (Corning, 3701) per experiment. The cultivation duration was seven days at 37° C. and 5.0% CO₂. The use of 384 well plates was possible because feeder cells are absent. It is an advantage of the novel cultivation method herein that smaller wells and cultivation volumes can be used.

An antigen specific ELISA was performed for each well to determine whether the anti-antigen IgG producing cell has proliferated. This is considered an indirect measure for cell proliferation.

The ELISA buffer contained 1.0% BSA (Roche, 3535240001) and PBS (Gibco, 10010-015). All steps were incubated at room temperature for 1 hour. The wash procedure included three wash steps with 300 μl wash buffer. The wash buffer contained 0.9% NaCl (Merck, 1064045000) and 0.05% Tween20 (Sigma Aldrich, 8.22184). 96-well (Roche, 11989685001) or 384-well (Roche, 11974998) Streptavidin precoated plates, which were incubated with a biotin labeled specific antigen with 200 ng/ml, were used. The antigen was the same as for the panning step in antigen specific B cell isolation. After the wash procedure the plates were incubated with the undiluted culture supernatants. After another wash procedure, the plates were incubated with a peroxidase (POD) labeled polyclonal goat anti-rabbit IgG-Fc in a 1:12.500 dilution. The dilution was done with ELISA buffer. Afterwards, the last wash procedure and the incubation with ABTS (Roche, 10102946001) for 10 minutes followed. The detection wave length was 405 nm and the reference wave length was 492 nm.

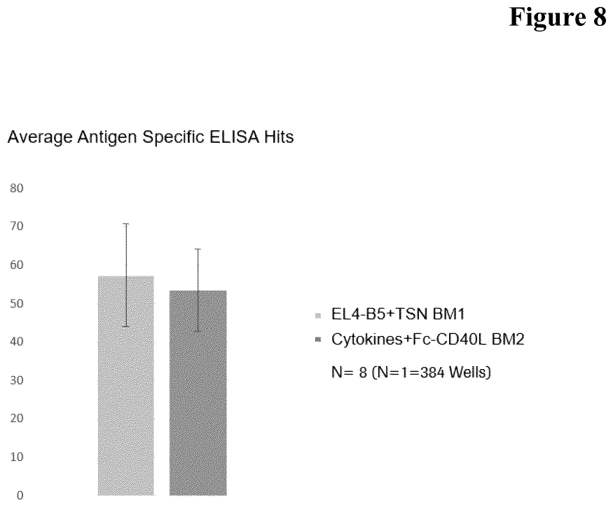

On average of 8 experiments with 384 wells per experiment, the reference setting with EL4-B5+TSN BM 1 exhibited with 57 antigen specific hits (FIG. 8). The four selected cytokines (IL-2, 11-15, IL-21 and BAFF)+Fc-CD40-L revealed 54 hits (i.e. wells having cells producing antigen-specific antibodies).

This finding shows that single cell culture conditions for antibody expressing cells could be established without the need for feeder cells and conditioned cell supernatant by using cytokines and a CD40-L-Fc protein.

1. An in vitro method for cultivating one or more antibody expressing cell(s) obtained from peripheral blood, said method comprising:
cultivating the one or more antibody expressing cell(s) in the presence of IL-2, IL-21 and a non-cell surface presented CD40-stimulating agent,
wherein said cultivating is performed in the absence of feeder cells.

2. The method of claim 1, wherein said cultivating of the one or more antibody expressing cell(s) is performed in the presence of IL-15 and/or BAFF.

3. The method of claim 1, wherein said cultivating of the one or more antibody expressing cell(s) is performed in the absence of conditioned cell supernatant.

4. The method of claim 1, wherein the one or more antibody expressing cell(s) is one antibody expressing cell, and wherein said cultivating is performed in a single cell format.

5. The method of claim 1, wherein the one or more antibody expressing cell(s) is/are one or more antibody secreting cell(s); and/or wherein the one or more antibody expressing cell(s) is/are primary cell(s).

6. The method of claim 1, wherein the one or more antibody expressing cell(s) are obtainable or obtained by a method comprising:
i) isolating PBMCs from peripheral blood;
ii) isolating from said PBMCs cells that are: a) IgG^{int} and b) have an intermediate cell size (FSC^{int}); and
iii) optionally, wherein the antibody expressing cells express an antibody specifically binding to an antigen of interest, and wherein the method by which these cells are obtained or obtainable further comprises isolating from the PBMCs cells binding to an antigen of interest.

7. The method of claim 1, wherein the antibody expressing cells are rabbit cell(s).

8. The method of claim 1, wherein the non-cell surface presented CD40-stimulating agent is a soluble CD40-stimulating agent.

9. The method of claim 1, wherein the non-cell surface presented CD40-stimulating agent is at least trimeric.

10. The method of claim 1, wherein the non-cell surface presented CD40-stimulating agent comprises a multimerization domain.

11. A method for producing an antibody directed to an antigen of interest comprising:
(i) cultivating one or more antibody expressing cell(s) obtained from peripheral blood according to the method as defined in claim 1, wherein all said one or more antibody expressing cell(s) express an antibody directed to the same antigen of interest; and
(ii) isolating the antibody from the culture medium and/or the antibody expressing cell(s).

12. A method for producing an antibody comprising:
(i) proliferating an antibody expressing cell obtained from peripheral blood using the cultivation method of claim 1;
(ii) determining the sequence of at least the heavy chain variable domain (VH) and the light chain variable domain (VL) of the antibody expressed by the antibody expressing cells obtained by the proliferation in step i);
(iii) expressing in a host cell an antibody comprising the heavy chain variable domain and the light chain variable domain encoded by the heavy chain variable domain sequence and the light chain variable domain sequence determined in (ii), respectively;
(iv) and isolating the expressed antibody.

13. A non-cell surface bound CD40-ligand (CD40-L) comprising (i) an CD40-L domain comprising or consisting of the amino acid sequence of SEQ ID NO: 1, 2, 3, 4, 5 or 6 or a variant thereof with CD40-L activity having at least 75%, at least 80%, at least 85%, at least 90%, at least 95% or at least 99% sequence identity thereto and (ii) optionally a multimerization domain.

14. (canceled)

15. A cell culture medium comprising a non-cell surface bound CD40-ligand (CD40-L) as defined in claim 13, IL-2 and IL-21 and optionally IL-15 and/or BAFF.

16. The method of claim 3, wherein the conditioned cell supernatant is a thymocyte supernatant (TSN).

17. The method of claim 5, wherein the one or more antibody secreting cell(s) are one or more plasmablast(s);

18. The method of claim 6, wherein the isolating in step ii) results from using flow cytometry.

19. The method of claim 8, wherein the soluble CD40-stimulating agent is a soluble CD40-ligand (CD40-L), wherein the non-cell surface presented CD40-stimulating agent comprises a CD40-L domain consisting of the amino acid sequence of SEQ ID NO: 1, 2, 3, 4, 5, or 6 or a variant thereof with CD40-L activity having at least 75%, at least 80%, at least 85%, at least 90%, at least 95%, or at least 99% sequence identity thereto.

20. The method of claim 9, wherein the non-cell surface presented CD40-stimulating agent is at least hexameric.

21. The method of claim 10, wherein the multimerization domain is selected from the group consisting of a Fc domain, a leucine zipper, and a C4b.


SEQUENCE LISTING (23 sequences)

SEQ ID NO: 1 - protein, 154 aa, Oryctolagus cuniculus
EINFEMQKGDQDPQIAAHLISEASSKSSSVLQWAKKGYYTMSNTLVTLENGKQLKVKRQGFYYIYAQVTFCSNQEPSSQAPFIASLCLKSSGGSERILLRAANARSSSKTCEQQSIHLGGVFELQADASVFVNVTDASQVNHGTGFTSFGLLKL

SEQ ID NO: 2 - protein, 154 aa, Homo sapiens
ENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 3 - protein, 154 aa, Rattus norvegicus
EKSFEMQRGDEDPQIAAHVVSEANSNAASVLQWAKKGYYTMKSNLVVLENGRQLTVKREGLYYVYTQVTFCSNREPLSQRPFIVSLWLKPSSGSERILLRAANTHSSSKLCEQQSIHLGGVFELQAGASVFVNVTEASQVIHGIGFSSIGLLKL

SEQ ID NO: 4 - protein, 154 aa, Mus musculus
ENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 5 - protein, 154 aa, Ovis aries
EKNFEMHKGDQEPQIAAHVISEASSKTTSVLQWAPKGYYTLSSNLVTLENGKQLAVKRQGFYYIYTQVTFCSNREALNQAPFIASLCLKSPSGSERILLRAANTHSSSKPCGQQSIHLGGVFELQQGASVFVNVTDPSQVSHGTGFTSFGLLKL

SEQ ID NO: 6 - protein, 163 aa, Cricetulus barabensis
IKLNTEEKKENSYEMQKGDEDPQIAVHVVSEANSKTASVLQWAKKGYYTMKNNLVMLESGKQLTIKRQGLYYVYTQVTFCSNQGPSSKDPFLVSLCLKSTSGSERILLRAANTHSSSKPCGQQSVHLGGVFELQEDSSLFVNVTDASQVIHGIGFTSFGLLKL

SEQ ID NO: 7 - protein, 163 aa, Oryctolagus cuniculus; identical to SEQ ID NO: 6

SEQ ID NO: 8 - protein, 58 aa, Homo sapiens
WETPEGCEQVLTGKRLMQCLPNPEDVKMALEVYKLSLEIEQLELQRDSARQSTLDKEL

SEQ ID NO: 9 - protein, 35 aa, synthetic construct
GDRMKQIEDKIEEILSKIYHIENEIARIKKLIGER

SEQ ID NO: 10 - protein, 22 aa, Saccharomyces cerevisiae
LEDKVEELLSKNYHLENEVARL

SEQ ID NO: 11 - protein, 244 aa, Homo sapiens
  VARIANT 243: note = No amino acid or G
  VARIANT 244: note = no amino acid or K
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGPRPPGPXX

SEQ ID NO: 12 - protein, 396 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGPRPPGPEINFEMQKGDQDPQIAAHLISEASSKSSSVLQWAKKGYYTMSNTLVTLENGKQLKVKRQGFYYIYAQVTFCSNQEPSSQAPFIASLCLKSSGGSERILLRAANARSSSKTCEQQSIHLGGVFELQADASVFVNVTDASQVNHGTGFTSFGLLKL

SEQ ID NO: 13 - protein, 267 aa, synthetic construct
GDRMKQIEDKIEEILSKIYHIENEIARIKKLIGERTSGGSGGTGGSGGTGGSHRRLDKIEDERNLHEDFVFMKTIQRCNKGEGSLSLLNCKEIRSQFEGFVKDIMLNKEEPKKEINFEMQKGDQDPQIAAHLISEASSKSSSVLQWAKKGYYTMSNTLVTLENGKQLKVKRQGFYYIYAQVTFCSNQEPSSQAPFIASLCLKSSGGSERILLRAANARSSSKTCEQQSIHLGGVFELQADASVFVNVTDASQVNHGTGFTSFGLLKL

SEQ ID NO: 14 - protein, 275 aa, synthetic construct
HHHHHHHHGDRMKQIEDKIEEILSKIYHIENEIARIKKLIGERTSGGSGGTGGSGGTGGSHRRLDKIEDERNLHEDFVFMKTIQRCNKGEGSLSLLNCKEIRSQFEGFVKDIMLNKEEPKKEINFEMQKGDQDPQIAAHLISEASSKSSSVLQWAKKGYYTMSNTLVTLENGKQLKVKRQGFYYIYAQVTFCSNQEPSSQAPFIASLCLKSSGGSERILLRAANARSSSKTCEQQSIHLGGVFELQADASVFVNVTDASQVNHGTGFTSFGLLKL

SEQ ID NO: 15 - protein, 187 aa, synthetic construct
LEDKVEELLSKNYHLENEVARLGGGSGGGSGGGEINFEMQKGDQDPQIAAHLISEASSKSSSVLQWAKKGYYTMSNTLVTLENGKQLKVKRQGFYYIYAQVTFCSNQEPSSQAPFIASLCLKSSGGSERILLRAANARSSSKTCEQQSIHLGGVFELQADASVFVNVTDASQVNHGTGFTSFGLLKL

SEQ ID NO: 16 - protein, 195 aa, synthetic construct
HHHHHHHHLEDKVEELLSKNYHLENEVARLGGGSGGGSGGGEINFEMQKGDQDPQIAAHLISEASSKSSSVLQWAKKGYYTMSNTLVTLENGKQLKVKRQGFYYIYAQVTFCSNQEPSSQAPFIASLCLKSSGGSERILLRAANARSSSKTCEQQSIHLGGVFELQADASVFVNVTDASQVNHGTGFTSFGLLKL

SEQ ID NO: 17 - protein, 223 aa, Homo sapiens
WETPEGCEQVLTGKRLMQCLPNPEDVKMALEVYKLSLEIEQLELQRDSARQSTLDKELGGGSGGGSGGGEINFEMQKGDQDPQIAAHLISEASSKSSSVLQWAKKGYYTMSNTLVTLENGKQLKVKRQGFYYIYAQVTFCSNQEPSSQAPFIASLCLKSSGGSERILLRAANARSSSKTCEQQSIHLGGVFELQADASVFVNVTDASQVNHGTGFTSFGLLKL

SEQ ID NO: 18 - protein, 231 aa, synthetic construct
HHHHHHHHWETPEGCEQVLTGKRLMQCLPNPEDVKMALEVYKLSLEIEQLELQRDSARQSTLDKELGGGSGGGSGGGEINFEMQKGDQDPQIAAHLISEASSKSSSVLQWAKKGYYTMSNTLVTLENGKQLKVKRQGFYYIYAQVTFCSNQEPSSQAPFIASLCLKSSGGSERILLRAANARSSSKTCEQQSIHLGGVFELQADASVFVNVTDASQVNHGTGFTSFGLLKL

SEQ ID NO: 19 - protein, 17 aa, synthetic construct
TSGGSGGTGGSGGTGGS

SEQ ID NO: 20 - protein, 20 aa, synthetic construct
MRRMQLLLLIALSLALVTNS

SEQ ID NO: 21 - protein, 19 aa, synthetic construct
MGWSCIILFLVATATGVHS

SEQ ID NO: 22 - protein, 11 aa, synthetic construct
GGGSGGGSGGG

SEQ ID NO: 23 - protein, 28 aa, synthetic construct
MGVHECPAWLWLLLSLLSLPLGLPVLGA